(19) United States
(12) Patent Application Publication
(10) Pub. No.: US 2024/0164895 A1
(43) Pub. Date: May 23, 2024

(54) Transcatheter Prosthetic Atrioventricular Valve with Stiffening Structure

(71) Applicant: St. Jude Medical, Cardiology Division, Inc., St. Paul, MN (US)

(72) Inventors: Son Mai, North Branch, MN (US); Heath Marnach, Minneapolis, MN (US); Randolf Von Oepen, Aptos, CA (US); William H. Peckels, Robbinsdale, MN (US)

(73) Assignee: St. Jude Medical, Cardiology Division, Inc., St. Paul, MN (US)

(21) Appl. No.: 18/508,404

(22) Filed: Nov. 14, 2023

Related U.S. Application Data
(60) Provisional application No. 63/384,521, filed on Nov. 21, 2022.

(51) Int. Cl. A61F 2/24

(52) U.S. Cl. CPC A61F 2/2418; A61F 2230/0017; A61F 2230/0063; A61F 2250/0069

(57) ABSTRACT

A prosthetic atrioventricular valve may include a collapsible frame including an atrial disk, a ventricular disk, and a center portion between the two. The frame may include commissure attachment features (“CAFs”) that include struts extending from the center portion. Prosthetic leaflets may be mounted to the CAFs. A sealing fabric may be coupled to the frame. A commissure support ring may couple to and extend around the plurality of CAFs. The atrial disk and the ventricular disk may each flare outwardly from the center portion of the frame. The center portion of the frame may define a minimum diameter of the frame. Each of the CAFs may be spaced from adjacent ones of the CAFs so that gaps in the frame are present between adjacent ones of the CAFs.

### CROSS-REFERENCE TO RELATED APPLICATIONS

This application claims priority to and benefit of the filing date of U.S. Provisional Patent Application No. 63/384,521, filed Nov. 21, 2022, the disclosure of which is hereby incorporated by reference herein.

### BACKGROUND OF THE DISCLOSURE

Heart valve disease is a significant cause of morbidity and mortality. One treatment for this disease is valve replacement. One form of replacement device is a bioprosthetic valve. Collapsing these valves to a smaller size or into a delivery system enables less invasive delivery approaches compared to conventional open-chest, open-heart surgery. Collapsing the implant to a smaller size and using a smaller delivery system minimizes the access site size and reduces the number of potential periprocedural complications.

The size to which an implant can be collapsed is limited by the volume of materials used in the implant, the strengths and shapes of those materials, and the need to function after expansion (or re-expansion). Using multiple steps and/or multiple delivery system devices may increase the time and complexity of a procedure.

Native atrioventricular valves (i.e., the tricuspid valve and the mitral valve) typically have a larger size and/or diameter compared to the native aortic valve and the native pulmonary valve. Among the native atrioventricular valves, a regurgitant tricuspid valve typically has a larger size and/or diameter than a regurgitant mitral valve. For example, for patients with severe tricuspid valve regurgitation, the diameter of the tricuspid valve may range from about 40 mm to about 66 mm, although these numbers are merely exemplary. As a result, prosthetic heart valve designs and considerations for replacing the different native heart valves are not identical. For example, to accommodate the large size of the mitral and tricuspid valve, recent prosthetic heart valve designs have included an outer frame with a large size to engage the native mitral or tricuspid annulus, and a smaller and generally cylindrical inner frame within that outer frame, the inner frame housing the prosthetic valve leaflets. However, this double-stented design generally increases the bulk of the prosthetic heart valve, resulting in a larger profile when collapsed within a delivery device. This, in turn, requires the delivery device (e.g., a catheter housing the collapsed prosthetic heart valve for delivery) to have a larger size to accommodate the large prosthetic heart valve. Typically, it is desirable for catheters of transcatheter heart valve delivery devices to have a smaller size, since the catheters may need to pass through the vasculature to reach the native heart valve in a minimally invasive manner. Thus, it would be desirable to have a prosthetic heart valve that is able to fit within a native tricuspid or mitral valve but is able to collapse to a small size to be accommodated in a relatively small profile delivery device.

### BRIEF SUMMARY OF THE DISCLOSURE

According to one aspect of the disclosure, a prosthetic heart valve for replacing a native atrioventricular valve includes a collapsible and expandable frame, the frame including an atrial disk, a ventricular disk, and a center portion extending between the atrial disk and the ventricular disk, the frame including a plurality of commissure attachment features that include struts that extend from the center portion of the frame. A plurality of prosthetic leaflets may be mounted to the plurality of commissure attachment features. A sealing fabric may be coupled to an outer surface of the frame. A commissure support ring may be coupled to and may extend around the plurality of commissure attachment features. In an expanded condition of the prosthetic heart valve, (i) the atrial disk and the ventricular disk may each flare outwardly from the center portion of the frame, (ii) the center portion of the frame may define a minimum diameter of the frame, and (iii) each of the plurality of commissure attachment features may be spaced from adjacent ones of the plurality of commissure attachment features so that gaps in the frame are present between adjacent ones of the plurality of commissure attachment features. The commissure support ring may be a collapsible and expandable structure that has a circular shape in an expanded condition of the commissure support ring. The commissure support ring may be a collapsible and expandable structure that has a lobed shape in an expanded condition of the commissure support ring. In the expanded condition of the commissure support ring, first portions of the commissure support ring aligned with the plurality of commissure attachment features may have a minimum diameter of the commissure support ring, and second portions of the commissure support ring aligned with middle portions of free edges of the plurality of prosthetic leaflets may have a maximum diameter of the commissure support ring. The commissure support ring may include a first circumferential row of generally diamond-shaped cells. The commissure support ring may include a second circumferential row of generally diamond-shaped cells adjacent the first circumferential row. The commissure support ring may include a plurality of connectors integrally formed with the commissure support ring, and the plurality of connectors may each have a shape that is complementary to a shape of each of the plurality of commissure attachment features. The frame may include a plurality of tines on the ventricular disk, each of the plurality of tines extending to a free end pointing toward the atrial disk in a collapsed condition of the frame. In the expanded condition of the prosthetic heart valve, at least some of the plurality of tines may extend at an acute angle relative to a central longitudinal axis of the prosthetic heart valve. In the expanded condition of the prosthetic heart valve, at least some of the plurality of tines may extend at an obtuse angle relative to a central longitudinal axis of the prosthetic heart valve. The struts that extend from the center portion of the frame may include at least one aperture. The commissure support ring may be coupled to the plurality of commissure attachment features via mechanical fasteners extending through the at least one aperture. In the expanded condition of the prosthetic heart valve, the ventricular disk of the frame may be bell-shaped. The sealing fabric may extend over the ventricular disk and over the center portion of the frame, and an inflow edge of the sealing fabric may be positioned a spaced distance from a terminal end of the atrial disk. In an implanted condition of the prosthetic heart valve, at least a portion of the sealing fabric may be configured to parachute into contact with structure of the native atrioventricular valve during ventricular systole.

According to another aspect of the disclosure, a method of implanting a prosthetic heart valve may include loading the prosthetic heart valve into a delivery device. The prosthetic heart valve may include a collapsible and expandable frame having an atrial disk, a ventricular disk, a center portion extending between the atrial disk and the ventricular disk, a plurality of commissure attachment features that include struts that extend from the center portion of the frame, and a plurality of prosthetic leaflets mounted to the plurality of commissure attachment features. The method may include advancing the delivery device to a native heart valve of a patient while the prosthetic heart valve is maintained in a collapsed condition by the delivery device, and while the delivery device is positioned in or adjacent to the native heart valve, starting to deploy the prosthetic heart valve in a ventricle of the patient so that the ventricular disk begins to expand and so that a sealing fabric coupled to an outer surface of the frame moves toward the native heart valve. The method may include continuing to deploy the prosthetic heart valve so that the center portion is positioned against an annulus of the native heart valve and the atrial disk expands within an atrium of the patient. While starting to deploy the prosthetic heart valve, a commissure support ring may be coupled to and may extend around the plurality of commissure attachment features to limit the distance which the plurality of commissure attachment feature can splay outwardly. While starting to deploy the prosthetic heart valve, at least one of a plurality of tines on the ventricular disk may frictionally engage tissue of the native heart valve. The commissure support ring may be a collapsible and expandable structure that has a circular shape after the prosthetic heart valve is fully deployed within the native heart valve. The commissure support ring may be a collapsible and expandable structure that has a lobed shape after the prosthetic heart valve is fully deployed within the native heart valve. After the prosthetic heart valve is fully deployed within the native heart valve, first portions of the commissure support ring aligned with the plurality of commissure attachment features may have a minimum diameter of the commissure support ring, and second portions of the commissure support ring aligned with middle portions of free edges of the plurality of prosthetic leaflets may have a maximum diameter of the commissure support ring, such that as the plurality of prosthetic leaflets open during atrial systole, the middle portions of the free edges of the plurality of prosthetic leaflets reach a position radially outward of the first portions of the commissure support ring and radially inward of the second portions of the commissure support ring.

### BRIEF DESCRIPTION OF THE DRAWINGS

FIG. 1 is a cross-section of a prosthetic heart valve, taken along section line 1-1 of FIG. 2.

FIG. 2 is a schematic side view of the prosthetic heart valve of FIG. 1.

FIG. 3 is a side view of the prosthetic heart valve of FIGS. 1-2.

FIG. 4 is a top or atrial view of the prosthetic heart valve of FIGS. 1-2.

FIG. 5 is a bottom or ventricular view of the prosthetic heart valve of FIGS. 1-2.

FIG. 6 is a perspective view of the stent of the prosthetic heart valve of FIGS. 1-2 with other components of the prosthetic heart valve omitted.

FIG. 7 is a view of a portion of the cut pattern of the stent of FIG. 6.

FIG. 8A is a perspective view of a prosthetic heart valve having a configuration different than the prosthetic heart valve of FIGS. 1-7.

FIG. 8B is a view of a portion of a cut pattern of a stent for use with the prosthetic heart valve of FIG. 8A.

FIG. 8C is a view of a cut pattern of an alternate version of a stent for use with the prosthetic heart valve of FIG. 8A.

FIG. 8D shows the prosthetic heart valve of FIG. 8A with certain structures omitted from the view for clarity.

FIG. 9A is a view of the prosthetic heart valve of FIG. 8A, without a commissure support structure, being deployed from a delivery device.

FIG. 9B is a view of the prosthetic heart valve of FIG. 8A, without a commissure support structure, being manually deformed.

FIGS. 10A-C show different cut patterns that may be used to form a commissure support structure for use with a prosthetic heart valve such as that shown in FIG. 8A.

FIG. 10D illustrates the commissure support structure of FIG. 10C overlaid on the frame of FIG. 8C.

FIG. 10E illustrates an alternate shape for a commissure support structure.

FIGS. 11A-B show a prosthetic heart valve with a commissure support structure prior to and after, respectively, application of manual compressive force to the prosthetic heart valve.

FIG. 12 shows the prosthetic heart valve, as represented in FIG. 8D, after having been implanted over a previously implanted leaflet repair device.

FIG. 13A is a view of a cut pattern of a frame having an alternate commissure attachment feature.

FIG. 13B is an enlarged view of the commissure attachment feature of the frame of FIG. 13A.

FIG. 13C is a view of a cut pattern that may be used to form an alternate commissure support structure.

FIG. 13D is an enlarged view of a portion of the cut pattern of FIG. 13C.

FIG. 13E illustrates the cut pattern of the commissure support structure of FIG. 13C overlaid on the cut pattern of the frame of FIG. 13A in a first orientation.

FIG. 13F illustrates the cut pattern of the commissure support structure of FIG. 13C overlaid on the cut pattern of the frame of FIG. 13A in a second orientation.

FIGS. 13G-H are views of the commissure support structure of FIG. 13C coupled to the frame of FIG. 13A, in an expanded condition.

### DETAILED DESCRIPTION OF THE DISCLOSURE

As used herein, the term inflow, when used in connection with a prosthetic heart valve, refers to the end of the prosthetic heart valve through which blood first flows when flowing in the antegrade direction, and the term outflow refers to the end of the prosthetic heart valve through which blood last flows when flowing in the antegrade direction. Further, although the disclosure focuses on prosthetic tricuspid valve replacements, the disclosure may apply with similar force to prosthetic mitral valve replacements. Thus, unless otherwise expressly specified, the embodiments described herein may be used for replacing either a native tricuspid valve or a native mitral valve (with or without additional modifications specific to the heart valve being replaced), even if a particular embodiment may be more suited for replacing either the native tricuspid valve or the native mitral valve.

As explained in the background of the disclosure, prosthetic heart valves that include an anchoring frame and a valve frame nested within the anchoring frame typically have larger sizes (e.g. larger crimped diameters) when collapsed within a delivery device. As an example, this type of prosthetic heart valve may only fit within a delivery device that has a catheter with an inner diameter that is 30-33 French (10-11 mm in diameter) or larger. For transcatheter prosthetic mitral or tricuspid valves that are delivered intravascularly through the femoral vein, a delivery catheter having an outer diameter of 30-33 French or larger may increase the likelihood of access site complications, which may require a surgeon to intervene, as well as requiring a surgical cut down to gain access to the vasculature and a follow-up surgical repair after the procedure is complete. The prosthetic heart valves disclosed herein have features and configurations that are intended to allow for the prosthetic heart valves to reliably anchor within the larger size annulus of the tricuspid valve (or the mitral valve) while being able to collapse into a delivery catheter having an inner diameter of 30-33 French or smaller. It should be understood that, as used herein, the unit French refers to the inner diameter of a catheter when describing the ability of a valve to fit within that catheter, whereas the unit French refers to the outer diameter of the catheter when describing how catheter size may result in vascular access problems.

As is described below, one way to achieve this functionality is to design the prosthetic heart valve with a single support stent (e.g., a single stent layer or a non-nested frame configuration) that can span the large atrioventricular valve annulus diameters found in patients who experience heart failure. The geometry of the support stent may allow for prosthetic leaflets to be secured inside, with atrial and/or ventricular flanges or disks that have a large enough diameter or profile to sandwich, clamp, or overlie the native annulus tissue therebetween. To provide adequate sealing between the support stent and the native valve annulus, fabric(s) may span the gap between the atrial and ventricular disks, where the fabric(s) are capable of elongating to mitigate the effects of foreshortening when sheathing the prosthetic heart valve into the catheter. While the embodiments described below may be suitable for replacing either a tricuspid or mitral valve, these embodiments may be best suited for replacing a tricuspid valve due to the lower right ventricular pressures compared to left ventricular pressures, which may reduce the need for a nested stent design. It should be understood that, although the terms “frame” and “stent” are generally used interchangeably herein, the term “stent” does not imply any special structure or function beyond being a frame.

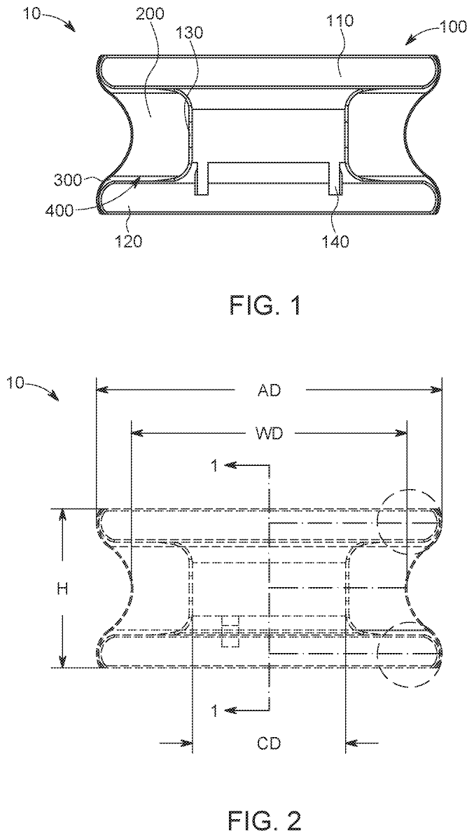

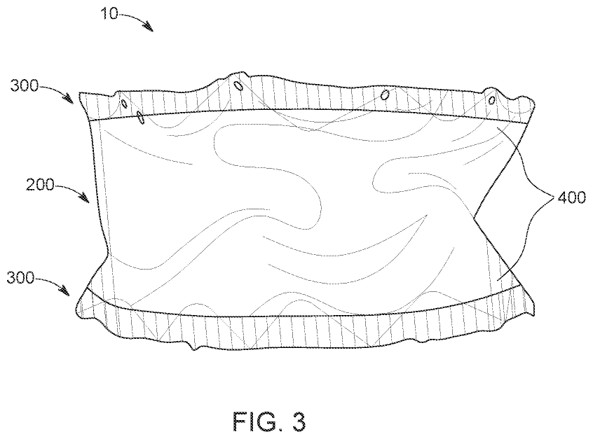

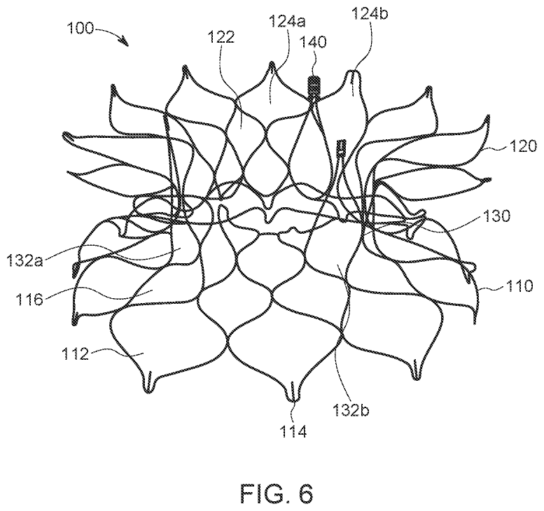

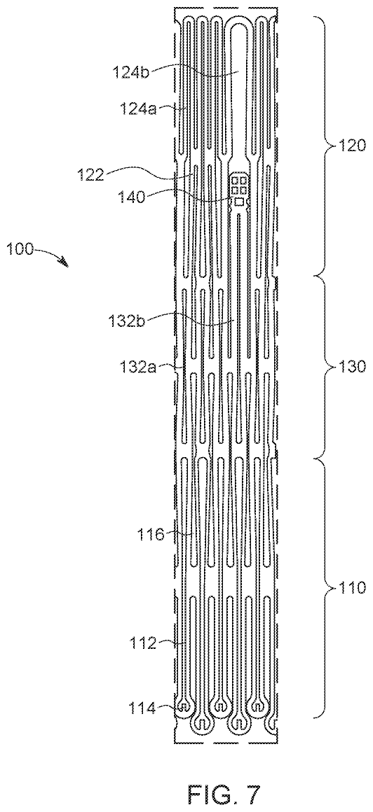

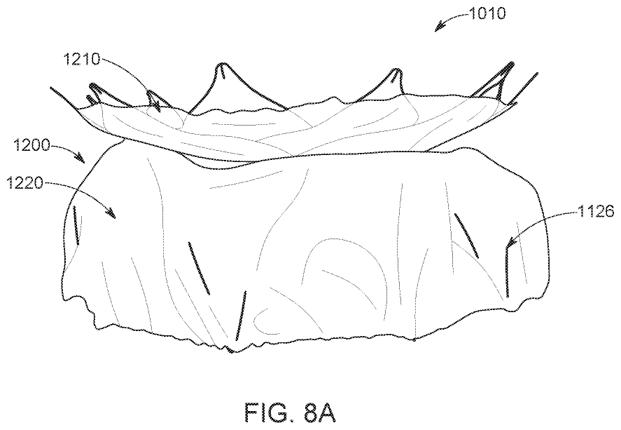

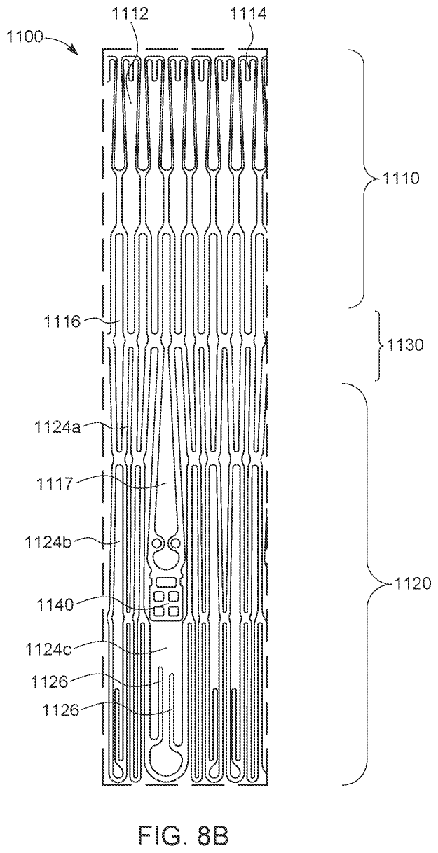

FIG. 1 is a cross-section of a prosthetic heart valve 10, taken along section line 1-1 of FIG. 2, according to one aspect of the disclosure. Generally, prosthetic heart valve 10 includes a support frame or stent 100, which may include an atrial flange 110 (which may alternately be referred to as an atrial disk or anchor), a ventricular flange 120 (which may alternately be referred to as a ventricular disk or anchor), and a central stent portion 130 (which may be referred to as a central waist). The stent 100 may also include a plurality of commissure attachment features (“CAFs”) 140 for use in coupling the prosthetic valve leaflets 500 to the stent 100. The prosthetic valve leaflets 500 are omitted from the illustration of FIGS. 1-2 and are best shown in FIGS. 4-5. The stent 100 is described in greater detail below in connection with FIGS. 6-7. Referring still to FIG. 1, the prosthetic heart valve 10 may include a first fabric 200 generally surrounding the central portion 130 of the stent 100 and spanning the gap between the atrial disk 110 and the ventricular disk 120. In one embodiment, the first fabric may be formed as a knitted fabric (e.g., polyethylene terephthalate (“PET”), polytetrafluoroethylene (“PTFE”), ultra-high molecular weight polyethylene (“UHMWPE”), polyester, or similar materials). The formation of the first fabric 200 as a knitted fabric may allow for significant stretching of the first fabric, e.g., up to doubling or tripling in length upon being stretched. In one embodiment, the first fabric 200 is configured to stretch to increase its length by a factor of about 2.5. When in the expanded or deployed condition shown in FIG. 1, a substantial amount of open space or volume may exist between the first fabric 200 and the outside of the stent 100, at least prior to implantation. A second fabric 300 may be provided on the outer surface of the atrial disk 110 and the ventricular disk 120, the second fabric 300 being coupled to the first fabric 200 at seams 400, which may be for example ultrasonic welding seams. In one embodiment, the second fabric 300 may be formed as a woven fabric (e.g., PET, PTFE, UHMWPE, etc.). By forming the second fabric 300 as a woven fabric, the second fabric 300 may be particularly suited to provide sealing functionality against the native anatomy, while the knitted fabric 200 is particularly suited to provide stretching capabilities to allow for relatively unhindered collapsing and expanding of the stent 100. Even if the first fabric 200 is formed as a knit fabric and the second fabric 300 is formed as a woven fabric, it may be desirable (although not required) for the two fabrics to be formed of the same material, with the stretch and/or sealing properties being influenced, at least in part, by the way in which the threads of the fabric are wound, knitted, and/or woven. For example, changing the bias of the warp versus the weft will change the stretch capabilities of a fabric.

Referring briefly to FIG. 2, various exemplary dimensions of the prosthetic heart valve 10 when the prosthetic heart valve 10 is in an expanded or deployed configuration are provided. However, these dimensions are merely exemplary and other dimensions may be suitable. For example, the outer anchor diameter AD of the atrial disk 110 and/or ventricular disk 120 may be about 65 mm. However, in other embodiments, the anchor diameter AD may be between about 50 mm and about 80 mm. In the expanded or deployed condition (e.g., upon implantation into the native tricuspid valve annulus), the first fabric 200 may form a waisted shape with a minimum waist diameter WD near an axial center of the first fabric 200. In the illustrated embodiment, the waist diameter WD may be about 52 mm, but in other embodiments, the waist diameter WD may be between about 45 mm and about 75 mm. The center portion 130 of the stent 100 may be generally cylindrical, and in the illustrated embodiment has a central diameter CD of about 29 mm, but in other embodiments, the central diameter CD may be between about 25 mm and about 35 mm. The prosthetic heart valve 10 may have a height H between the inflow and outflow ends of about 30 mm, but in other embodiments, the height may be between about 25 mm and about 45 mm. As noted above, each of these dimensions is merely illustrative. In some embodiments, in the expanded or deployed condition, the ratio of the outer anchor diameter AD to the central diameter CD is between about 2.5:1 and about 1.5:1, preferably between about 2.5:1 and about 2.0:1, including about 2.25:1. As is explained in greater detail below, this large disparity may allow the atrial disk 110 and ventricular disk 120 to be large enough to provide anchoring, but the central portion 130 to be small enough to house an appropriately sized set of prosthetic valve leaflets 500, while still maintaining a small crimp profile for delivery, e.g. capable of being delivered within, and deployed successfully from, a delivery device catheter having an inner diameter as small as 24 French (8.0 mm) or even as small as 18 French (6.0 mm). In some embodiments, the prosthetic heart valve 10 may be successfully housed within a catheter having an inner diameter of between 22 French (7.33 mm) and 32 French (10.66 mm), including between about 24 French (8.0 mm) and 28 French (9.33 mm).

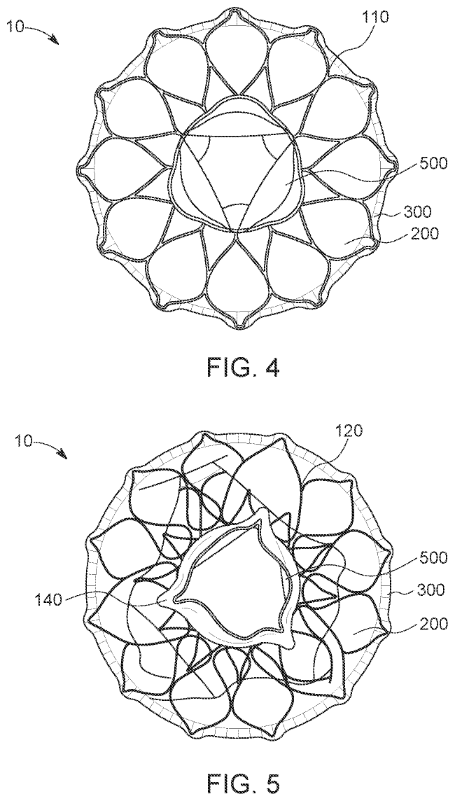

FIG. 3 is a side view of the prosthetic heart valve 10. FIG. 3 illustrates the prosthetic heart valve 10 in an expanded or deployed condition, with the first fabric 200 extending between the atrial disk 110 and the ventricular disk 120. In the illustrated embodiment, first fabric 200 is a stretchy fabric that can easily elongate when the prosthetic heart valve 10 collapses and foreshorten as the prosthetic heart valve 10 expands. To achieve this elongation and foreshortening, it is preferred that the first fabric 200 is not directly coupled to the stent 100. For example, the fabric 200 preferably is not directly sutured to struts of the stent 100. The second fabric 300 includes a portion coupled to the outside of the atrial disk 110 and a portion coupled to the outside of the ventricular disk 110, for example by suturing. As noted above, the second fabric 300 may function to assist with creating or enhancing the seal between the native valve annulus and the atrial and ventricular portions of the prosthetic heart valve 10. The interfaces 400 between the first fabric 200 and the portions of the second fabric 300 are shown in FIG. 3. In the illustrated example, the interfaces 400 are seams created via ultrasonic welding, but the fabrics may be coupled to each other in any suitable way, including suturing, adhesives, etc. In some embodiments, only a single outer fabric may be used, with the single outer fabric having sufficient stretch capabilities to allow the stent 100 to expand and collapse without significant restriction, but while also providing suitable sealing with the native valve annulus.

FIG. 4 illustrates the prosthetic heart valve 10 in an expanded or deployed condition, as viewed from the atrial or inflow side with the prosthetic leaflets 500 in an open condition. FIG. 5 illustrates the prosthetic heart valve 10 in an expanded or deployed condition, as viewed from the ventricular or outflow side with the prosthetic leaflets 500 in an open condition. In the illustrated embodiment, the prosthetic heart valve 10 includes three prosthetic leaflets 500, but in some embodiments, the prosthetic heart valve 10 may include more or fewer prosthetic leaflets. The prosthetic leaflets 500 may be formed of any suitable material. For example, each prosthetic leaflet 500 may be formed of bioprosthetic tissue, such as porcine or bovine pericardium. In other embodiments, each prosthetic leaflet 500 may be formed of a synthetic material such as a synthetic material or fabric, including PET, UHMWPE, PTFE, etc. Each prosthetic leaflet 500 may have two side portions (e.g., forming leaflet tabs), each side portion being coupled to an end of an adjacent prosthetic leaflet 500 to form a commissure, each leaflet commissure being coupled to a respective CAF 140 of the stent 100. Each leaflet may include an outflow edge between the two side edges, the outflow edge being free to allow for opening and closing of the leaflets during normal operation (e.g., to provide the valve functionality). Each leaflet may include an inflow edge between the two side edges, the inflow edge being coupled (e.g., sutured) to the stent 100. In some embodiments, the inflow edges of the prosthetic leaflets 500 are coupled solely (e.g., via sutures) to struts of the central portion 130 of the stent 100, without any direct attachment to other components. In other embodiments, a short fabric skirt may be provided around a portion of the inflow side of the central portion 130 of the stent 100, with the short fabric skirt coupled to the stent 100 and providing an additional structure that may be used for coupling (e.g., via sutures) the inflow ends of the prosthetic leaflets 500 to the prosthetic heart valve 10.

By comparing FIG. 4 to FIG. 5, it can be seen that the atrial disk 110 is “closed,” while the ventricular disk 120 is “open.” In other words, as shown in FIG. 4, the inflow ends of the prosthetic leaflets 500 are directly coupled to the stent 100 (and/or a separate short fabric skirt as noted above) so that there is no open flow pathway between the prosthetic leaflets 500 and the outer fabric skirt(s) (e.g., first fabric 200 and second fabric 300, or the single outer fabric if only a single outer fabric is used) at the atrial disk 110. On the other hand, as shown in FIG. 5, the outflow ends of the prosthetic leaflets 500 have a significant radial spacing from the first fabric 200 and the second fabric 300. With this configuration, after implantation of the prosthetic heart valve 10 into the native tricuspid valve, as the right ventricle contracts, the prosthetic leaflets 500 are forced closed and blood tends to flow in the retrograde direction into the space between the prosthetic leaflets 500 and the first fabric 200. The “closed” configuration at the atrial disk 110 ensures that blood cannot actually pass into the right atrium, but the “open” configuration at the ventricular disk 120 allows that retrograde blood to press the first fabric 200 outwardly and into the native valve annulus to enhance the seal between the prosthetic heart valve 10 and the native tricuspid valve annulus.

FIG. 6 is a perspective view of the stent 100 of the prosthetic heart valve 10 in an expanded condition, with other components of the prosthetic heart valve 10 omitted from the figure. The orientation of stent 100 is opposite that shown in FIGS. 1-3. In other words, the top of the view of FIG. 6 is the outflow end and the bottom of the view of FIG. 6 is the inflow end. Stent 100 is preferably formed of a biocompatible shape memory or superelastic material. One particularly suitable example of this stent material is a nickel-titanium alloy, such as Nitinol. However, other materials may be suitable. In one example, stent 100 may be formed by laser cutting a hollow tube of Nitinol, and then shape-setting the stent 100 to the desired shape, for example by heat treatment. With this configuration, the stent 100 may take the set shape, such as that shown in FIG. 6, in the absence of applied forces. To deliver the prosthetic heart valve 10, the prosthetic heart valve 10 may be collapsed to a small diameter and positioned within a delivery catheter to be passed intravascularly through the patient into the patient's heart. FIG. 7 shows the cut pattern of a portion of stent 100.

Referring collectively to FIGS. 6-7, the stent 100 may be formed with a plurality of rows of generally diamond-shaped cells. In the illustrated example, the atrial disk 110 includes an inflow row of cells 112, which may include a total of twelve cells. A pin 114 may be formed at the inflow apex of one, some, or each cell 112, the pin extending a short distance in the outflow direction to a free end. Each pin 114 may be sized and shaped so that a suture loop of the delivery device may slip over the pin 114, keeping the stent 110 connected to the delivery device during delivery and deployment. Upon deployment of the prosthetic heart valve 10, each suture loop may be pushed forward or distally to disengage with the corresponding pins 114 to fully decouple the prosthetic heart valve 10 from the delivery device. Similar pins and suture loops are described in more detail in U.S. Pat. No. 10,874,512, the disclosure of which is hereby incorporated by reference herein.

A transition row of cells 116 may be positioned directly adjacent to the inflow row of cells 112, with cells 116 transitioning between the atrial disk 110 and the central portion 130. In the illustrated embodiment, a total of twelve atrial transition cells 116 are provided. The central portion 130 may be generally formed by a row of central cells, although each central cell may not be identical to each other central cell. In the illustrated example, there are a total of twelve central cells, with a total of nine connected central cells 132a, and three free central cells 132b. Each connected central cell 132a may be diamond-shaped and have both inflow and outflow apices of the central cell 132a connected directly to another cell of the stent. Each free central cell 132b may have an inflow apex coupled directly to the outflow apex of an inflow cell 112, with the outflow apex of the free central cell 132b transitioning into a CAF 140. With this configuration, there are three free central cells 132b evenly spaced around the circumference of the stent 100, and three consecutive connected central cells 132a positioned between each pair of free central cells 132b. In the illustrated embodiment, each CAF 140 is cantilevered in the sense that it is only coupled to the stent 100 on one side, via two struts of the corresponding free central cell 132b. This cantilevered configuration may allow for greater leaflet height while maintaining a relatively small distance between the atrial disk 110 and the ventricular disk 120. The cantilevered configuration may also allow for greater deflection of the CAFs 140 during normal operation of the prosthetic heart valve 10, which may reduce stress on the prosthetic leaflets 500. Each CAF 140 may include one or more eyelets to assist in suturing or otherwise coupling the commissures of the prosthetic leaflets 500 to the CAFs 140. Each CAF 140 may also include a separate fabric coupled to the CAF 140 to assist with suturing of the prosthetic leaflets 500 to the CAF 140. In the illustrated embodiment, each CAF 140 includes a two-by-two array of four small eyelets, with a larger eyelet positioned between the array of four small eyelets and the two struts of the free central cell 132b. However, other configurations of eyelets and other types of CAFs may be suitable as an alternative to CAF 140.

Still referring to FIGS. 6-7, a row of ventricular transition cells 122 may be positioned directly adjacent to the central cells. In the illustrated embodiment, there are a total of six ventricular transition cells 122 provided in pairs, with each ventricular transition cell 122 positioned between two adjacent connected central cells 132. In this example, there are no ventricular transition cells 122 directly adjacent to the free central cells 132b. The ventricular transition cells 122 may form part of the cylindrical central portion 130, and flare outwardly to form part of the ventricular disk 120. In the illustrated embodiment, the final ventricular row of cells includes small ventricular cells 124a and large ventricular cells 124b. For example, a total of nine small ventricular cells 124a may be provided, each with an inflow apex directly connected to the outflow apex of a connected central cell 132a. Between each series (e.g., series of three) small ventricular cells 124a, a large ventricular cell 124b is positioned. Portions of the free central cells 132b, including the CAFs 140, may nest within the large ventricular cell 124b when the stent 100 is collapsed. Thus, in the illustrated embodiment, a total of three large ventricular cells 124b are provided. Although one specific configuration of cells is shown and described in connection with FIGS. 6-7, it should be understood that other configurations of cells may provide for suitable functionality of the prosthetic heart valve 10. With the embodiment described above, the stent 100 may be formed as a single unitary or monolithic structure that, when expanded or deployed, has an hourglass-type shape with two opposite disks having a large diameter for anchoring on the atrial and ventricular sides of the native annulus, with a generally cylindrical center portion extending therebetween having a significantly smaller diameter than the disks. As is explained in greater detail below, the central portion 130 of the stent 100 may be positioned a spaced distance from the native annulus after implantation, which is generally in contrast to typical valves where there is stent structure pushing directly against the native annulus tissue after the prosthetic heart valve is implanted. Rather, as described below, the first fabric 200 directly contacts the native annulus tissue upon implantation, with space remaining between the first fabric 200 and the central portion 130 of the stent 100. By avoiding having two separate stents that overlap and increase the bulk of the prosthetic heart valve when collapsed into a delivery device, prosthetic heart valve 10 may be collapsed into a catheter for delivery, the catheter having an inner diameter of smaller than 30 French, including for example 28 French or 24 French or even smaller. Other benefits of this single stent design may include lower cost, and feasible retrieval of the prosthetic heart valve, as a connector between two nested stents might make retrievability more difficult compared to a single stent design.

Referring briefly back to FIGS. 1-3, to allow for the stent 100 to collapse into the delivery device and then expand upon deployment, the outer fabric is preferably designed to not overly restrict the stent 100 from collapsing and expanding. For example, sealing fabrics provided on stents of prosthetic heart valves are frequently woven fabrics and are often tightly sutured or otherwise coupled to the stent so that the fabric does not have the ability to significantly stretch or deviate from its position relative to the stent. Further, these fabrics are often designed with sealing against blood flow as a primary fabric characteristic. On the contrary, prosthetic heart valve 10 has an outer fabric that is capable of significant stretching to accommodate the change in the shape of the stent 100 during transitioning between the collapsed and expanded conditions, with the fabric being spaced a significant distance from the central portion 130 when the prosthetic heart valve 10 is deployed. For example, referring to FIG. 1, when the prosthetic heart valve 10 is deployed into the native valve annulus, the atrial disk 110 and ventricular disk 120 may generally wrap around the atrial and ventricular sides of the native valve annulus, with the outer fabric pressing against the native valve annulus (e.g., via systolic pressure against the fabric on the outflow side), and the central portion 130 of the stent 100 generally centered within (but not directly pressing against) the tissue of the valve annulus. This configuration may allow the central portion 130 of the stent 100 to be a smaller size (e.g., about 28 mm to 32 mm) which may be optimal for hemodynamics, without needing to add additional stent structure to allow for proper sealing and/or anchoring in the much larger sized tricuspid valve annulus. This, in turn, may allow for the prosthetic heart valve 10 to collapse down to a small size for loading into a relatively small delivery device to minimize the likelihood of procedural complications (in particular vascular access complications) resulting from the size of the delivery device. The outer fabric may be formed as a single piece of knit fabric that allows for stretching in one direction, with the ability to seal against the native anatomy and for blood to quickly clot into the fabric to reduce the likelihood of paravalvular (“PV”) leak past the prosthetic heart valve 10 after implantation. However, in other embodiments, such as shown in FIG. 1, the outer fabric may include a first fabric 200 that is formed of a material chosen for its ability to stretch (e.g. a knit fabric), and a second fabric 300 at the atrial disk 110 and ventricular disk 120 that is chosen for the sealing, ingrowth, and/or clotting properties (e.g. a woven fabric), with the first fabric 200 coupled to the second fabric 300 in any desired fashion that allows the stretching to occur.

Although prosthetic heart valve 10 is described above with a particular frame or stent 100, and a particular configuration of sealing fabrics (e.g., first fabric 200 and second fabric 300), it should be understood that these are merely exemplary configurations and other configurations may be suitable. For example, additional configurations of the frame and sealing fabric are described in greater detail in U.S. Provisional Patent Application No. 63/341,702, filed May 13, 2022 and titled “Transcatheter Valve—Single Stent Structure With Fabric,” the disclosure of which is hereby incorporated by reference herein.

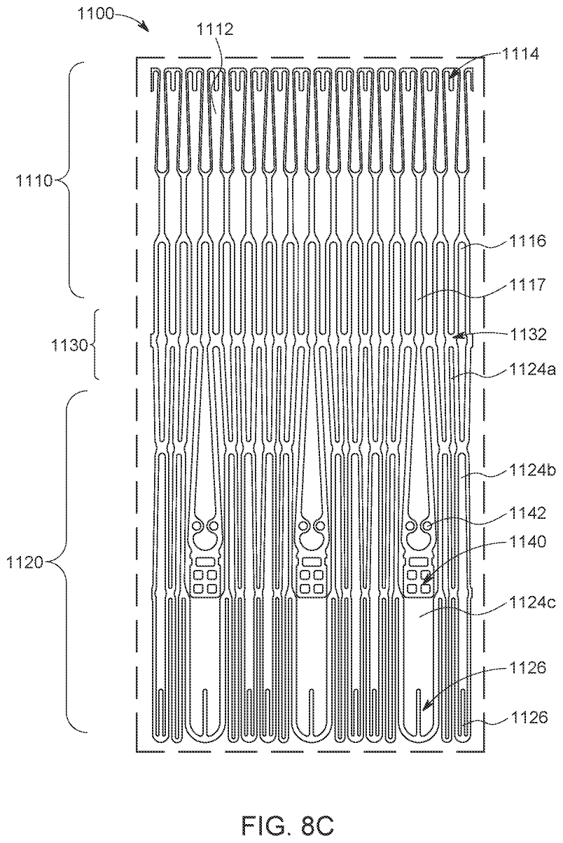

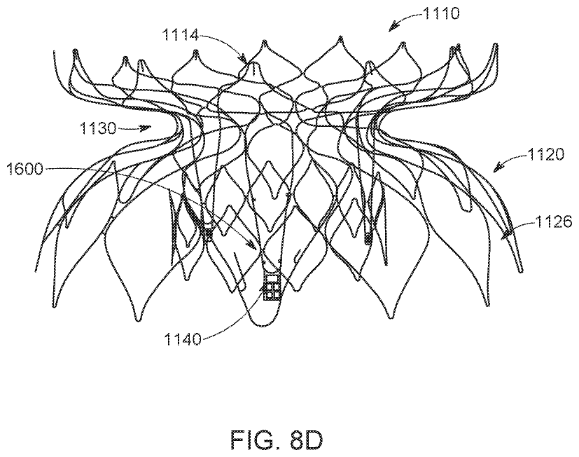

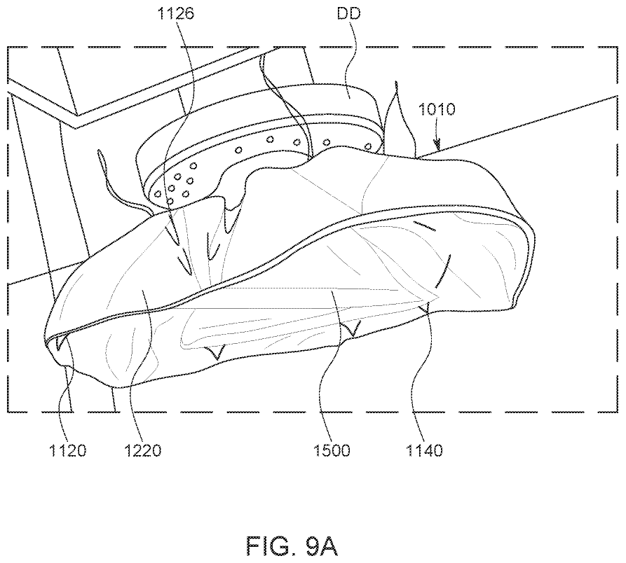

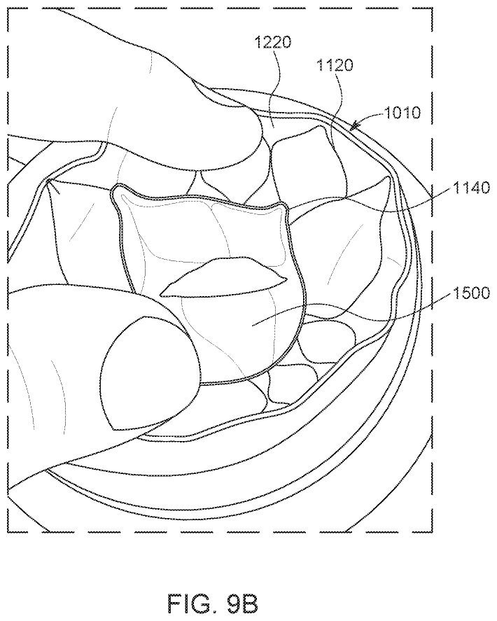

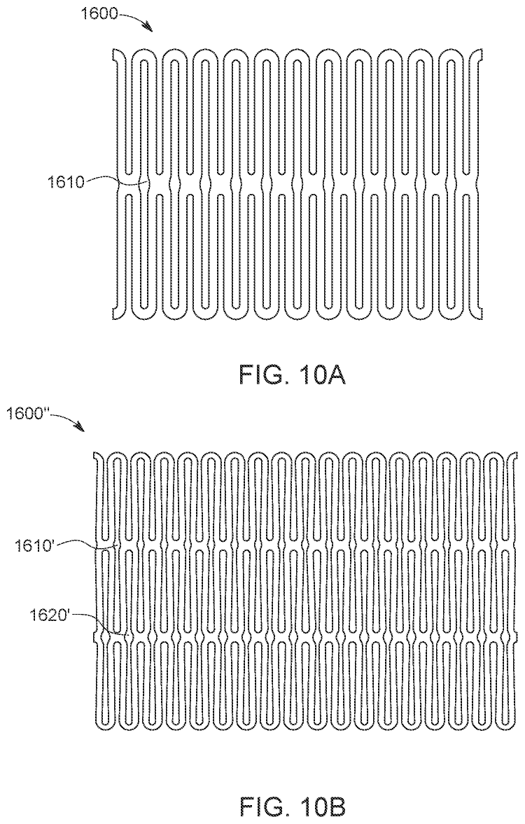

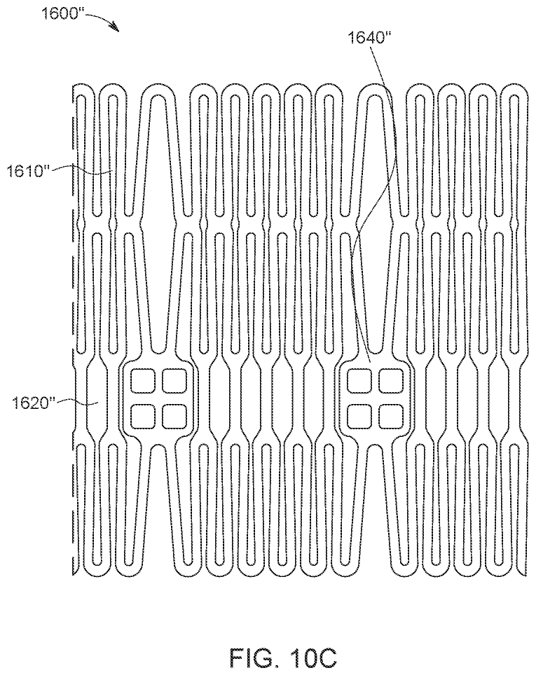

FIG. 8A illustrates a prosthetic heart valve 1010 according to another embodiment of the disclosure. As with prosthetic heart valve 10, prosthetic heart valve 1010 may be particularly suited for replacing a native atrioventricular valve, and in particular the native tricuspid valve. Prosthetic heart valve 1010 may include four components generally, including a stent or frame 1100, a sealing skirt 1200, prosthetic leaflets 1500 (shown in FIGS. 9A-B), and a commissure support member 1600 (shown in FIG. 8D), which may also be referred to as a stiffening structure. Prosthetic heart valve 1010 is shown in FIG. 8A in an expanded or deployed condition and is oriented with the atrial or inflow end of the valve toward the top of the view of FIG. 8A.

FIG. 8B illustrates a portion of a cut pattern of a stent of frame 1100 that may be used with prosthetic heart valve 1010. FIG. 8C illustrates a cut pattern of a stent or frame 1100 that may be used with prosthetic heart valve 1010. Although the frames of FIGS. 8B-8C have minor differences, the frames are highly similar and thus the same part numbers are used for the common features between the frames.

In FIGS. 8B-C, the frame 1100 has the same orientation as shown in FIG. 8A. In other words, in the views of FIGS. 8B-C, the inflow or atrial end of the frame 1100 is oriented toward the top of the view. Frame 1100 is preferably formed from a shape-memory material, such as a nickel-titanium alloy such as Nitinol, and may be created from a single tube, for example via laser cutting a tube of Nitinol. In the cut patterns shown in FIGS. 8B-C, the frame 1100 generally includes an atrial portion 1110 and a ventricular portion 1120 separated by a center portion 1130. After the frame 1100 is cut and set to the desired shape, for example as shown and described in greater detail in connection with FIG. 8D, the center portion 1130 may be very short, particularly in comparison to the center portion 130 of frame 100 shown in FIGS. 1-2.

Frame 1100 may include an atrial-most or inflow-most row of atrial cells 1112, which may be generally diamond-shaped cells that, in the expanded condition, flare radially outwardly from the center portion 1130. A pin 1114 may be formed at the inflow apex of one, some, or each atrial-most cell 1112, the pin 1114 extending a short distance in the outflow direction to a free end. Each pin 1114 may be sized and shaped so that a suture loop of the delivery device may slip over the pin 1114, keeping the frame 1100 connected to the delivery device during delivery and deployment. Upon deployment of the prosthetic heart valve 1010, each suture loop may be pushed forward or distally to disengage with the corresponding pins 1114 to fully decouple the prosthetic heart valve 1010 from the delivery device. Similar pins and suture loops are described in more detail in U.S. Pat. No. 10,874,512, the disclosure of which is hereby incorporated by reference herein. The atrial cells 1112 may terminate, at their outflow ends, at an inflection point 1132 (labeled in FIG. 8C). When the frame 1100 is shape-set to the desired shape, which may be generally similar to that shown in FIG. 8D, the inflection points 1132 may define the smallest diameter of the center portion 1130. It should be understood that the term “inflection point” is not necessarily used according to its mathematical definition, but rather references the point at which the frame 1100 changes from decreasing diameter to increasing diameter.

Still referring to FIGS. 8B-C, a plurality of transition cells 1116, which may be generally diamond-shaped, may be positioned in a row that is adjacent to the atrial cells 1112 in the outflow direction. Transition cells 1116 may include an inflow portion on the inflow side of center portion 1130 and an outflow portion on the outflow side of center portion 1130. In some examples, the transition cells 1116 may be axially centered about the inflection point 1132. The row of transition cells 1116 may include three enlarged transition cells 1117 (or more or fewer than three depending on the number of prosthetic leaflets included in the prosthetic heart valve 1010) that terminate in a commissure attachment feature (“CAF”) 1140. Preferably, the enlarged transition cells 1117 are positioned at substantially equal circumferential intervals around the frame 1100. As best shown in FIG. 8C, the sides of the atrial cells 1112 (which may extend to the inflow apex of the transition cells 1116 and the enlarged transition cells 1117) may include elongated beams 1115. These elongated beams 1115 may provide additional flexibility to the atrial portion 1110 (which may be referred to as the atrial disk). For example, depending on the numbers of cells included in the atrial portion, and the desired diameter that the atrial portion will span, the length of the beams 1115 may be adjusted. As the desired diameter of the atrial portion 1110 increase, the length (in the axial direction) of the diamond-shaped cells that form the atrial portion 1110 may need to increase if a particular opening angle (e.g., about 90 degrees) of the diamond-shaped cells is desired. As the axial length of the diamond-shaped cells increase in the differently-sized valve frames, the beams 1115 may correspondingly increase or decrease in length. However, in some embodiments, the beams 1115 may be omitted and the atrial row of cells 1112 may all be “full” diamond-shaped cells.

Each CAF 1140 may serve as an attachment point to the prosthetic leaflets 1500 (shown in FIGS. 9A-B, 11A-B). For example, each CAF 1140 may include a plurality of holes, and sutures may be used to couple adjacent pairs of leaflets to the CAFs 1140 via the holes therein. While CAFs 1140 are shown with four small holes in a two-by-two configuration and an elongated hole, other specific CAF configurations may be suitable for use instead of those shown.

The portion of the frame 1100 in the outflow direction of the inflection point 1132 may include a plurality of ventricular cells. For example, a group of first ventricular cells 1124a which may be generally diamond-shaped cells, the inflow apex of which is an inflection point 1132. A group of second ventricular cells 1124b may extend to the outflow-most portion of the frame 1100, the inflow apices of the second ventricular cells being connected to the outflow apices of the transition cells 1116. Some, none, or all of the second ventricular cells 1124b may include tines 1126, described in greater detail below, that may act as frictional engagement members that frictionally engage native tissue for enhancing securement of the frame 1100 within the native valve annulus. A group of third ventricular cells 1124c may be positioned between certain pairs of second ventricular cells 1124b, and may include struts that extend from the inflection point 1132 to the terminal outflow end of the ventricular portion 1120. Third ventricular cells 1124c may be larger than the other ventricular cells and may be formed in part by the struts of enlarged transition cells 1117 that terminate at CAFs 1140. With this configuration, at least in the cut pattern shown in FIGS. 8B-C, the CAFs 1140 may be thought of as either nested within third ventricular cells 1124c or forming a boundary of third ventricular cells 1124c.

In addition to tines 1126 being positioned in some, none, or all of the second ventricular cells 1124b, none, some, or all of the third ventricular cells 1124c may include tines 1126. The only difference between the frames shown in FIGS. 8B-C are the position and configuration of the tines. In the embodiment shown in FIG. 8B, the third ventricular cells 1124c each has two tines 1126, with one tine 1126 extending upward from each of the two outflow-most struts forming the cell. In FIG. 8C, on the other hand, each third ventricular cell 1124c includes a single tine 1126 extending upward from an outflow apex of the cell. In the embodiment shown in FIG. 8B, only some of the second ventricular cells 1124b include tines 1126, such that each second ventricular cell 1124b that includes a tine 1126 includes a single tine 1126 extending upward from one of the outflow struts near an outflow apex of the cell. In FIG. 8C, on the other hand, only some of the second ventricular cells 1124b include tines 1126, such that each second ventricular cell 1124b that includes a tine 1126 includes a single tine 1126 extending upward from an outflow apex of the cell. All of the tines 1126 may extend to a free tip that may have a sharp or blunt point, that is intended either to pierce tissue or to frictionally engage the tissue without piercing it. It should be understood that the number and positioning of the tines 1126 may be different from those shown in FIGS. 8B-C, and the specific number and positioning shown in FIGS. 8B-C is merely exemplary.

In the illustrated embodiments, the tines 1126 may be connected at an outflow end of the tine, with the free tip being positioned at an inflow end of the tine. This directionality of tines, compared to the tines being connected at their inflow end and having free tips at their outflow ends, may allow for a smoother and easier deployment of the valve from the delivery catheter. In other words, as the valve begins to self-expand as it is released from the delivery catheter, the tines do not begin to expand until the entire tine is free of the delivery device. With the opposite orientation, the tines might otherwise begin to extend radially outwardly and into contact with the end of the delivery sheath, which might make deployment more difficult. However, it should be understood that the illustrated directionality of tines may make the loading process slightly more difficult compared to the opposite directionality. However, smooth and easy deployment is typically more important than smooth and easy loading, and the loading process can be highly controlled and is performed outside the patient, while the deployment process is performed inside the patient.

After forming the frame 1100 by using the cut pattern shown in FIG. 8B or 8C, or another generally similar cut pattern, the frame 1100 may be shape-set, for example via heat treatment, to the desired shape. FIG. 8D illustrates one example of a frame 1100 that has a cut pattern similar to that shown in FIGS. 8B-C, after having been shape set and having been connected to a commissure support 1600, described in greater detail below.

As can be seen in FIG. 8D, when the frame 1100 is in the expanded or deployed condition the bottom of the atrial portion 1110 may be substantially straight with a slight upward angle, with the top half of the atrial portion 1110 may flare upwardly so that the tips of the atrial cells 1112 point generally in the inflow direction. The contours described above may be other than as exactly described while still being suitable for use in the prosthetic heart valve 1010.

Still referring to FIG. 8D, the ventricular portion 1120 may form a general “bell” shape with a more rounded and less flat contour compared to the atrial portion 1110. The more gentle contour of the ventricular portion 1120 may allow for the ventricular portion 1120 to drape against the ventricle and apply only light pressure to assist in fixing or otherwise securing the prosthetic heart valve 1010 to the native valve annulus. This light pressure or draping may be a first mechanism by which the prosthetic heart valve 1010 achieves fixation within the native valve annulus.

The various tines 1126 described above may be shape set so that the free ends of the tines 1126 are positioned away from the surfaces defined by the cell in which the tine 1126 is located. In other words, the tines 1126 may be bent or shaped so that the tips are available to pierce tissue or to frictionally engage tissue without piercing to provide a second mechanism by which the prosthetic heart valve 1010 achieves fixation within the native valve annulus. The tines 1126 may be oriented at different angles to achieve different objectives. For example, in some embodiments, some or all of the tines 1126 may be oriented or angled with the free ends pointing toward the atrial portion 1110 at an acute angle relative to the longitudinal axis passing through the center of the prosthetic heart valve 1010. Tines 1126 pointing at an acute angle, compared to a right angle or an obtuse angle, may be less likely to perforate tissue at the native valve annulus. Patients that may be in need of a prosthetic atrioventricular valve, particularly a prosthetic tricuspid valve, may be likely to have very thin medial walls in the ventricle, and acutely angled tines 1126 may particularly reduce the likelihood of the medial wall getting perforated by the tines 1126. There may be additional benefits to having an acutely angled tine 1126 compared to tines 1126 with larger angles (e.g., right angle or obtuse angle), relating to loading and deployment of the prosthetic heart valve 1010. For example, if the tines 1126 are more acutely angled, they may provide less resistance when the prosthetic heart valve 1010 is loaded into, or deployed from, the delivery catheter. Less resistance may equate to a more manageable load, which—all else being equal—may allow for a smaller size delivery catheter to be used. However, this is just one option. Some or all of the tines 1126 may instead be shape set to be oriented more laterally, for example a relatively large acute angle, or a right or obtuse angle, relative to the central longitudinal axis of the prosthetic heart valve 1010. Although the tines 1126 may be optional entirely, if the tines 1126 are included, whether they are acutely or laterally oriented, the tines 1126 may provide a second mechanism by which the prosthetic heart valve 1010 achieves fixation within the native valve annulus.

Before describing the support member 1600 in more detail, an exemplary sealing skirt 1200 that may be used with the prosthetic heart valve 1010 is described. Referring to FIG. 8A, an outer sealing skirt 1200 may be provided on the exterior of the frame 1100. In some embodiments, the sealing skirt 1200 may be the same as or similar to any of the embodiments described above. In the particular example shown in FIG. 8A, the sealing skirt 1200 may be a single piece of material (although in some embodiments it may be a multi-piece design), which may be any of the materials described in connection with the sealing skirts above, including for example woven PET. In the illustrated embodiment of FIG. 8A, the sealing skirt 1200 may have an atrial skirt portion 1210 and a ventricular skirt portion 1220. The atrial skirt portion 1210 may be coupled to the atrial portion 1110 of the frame 1100 with a relatively tight connection—for example via suturing along the struts of the atrial portion 1110 of the frame 1100. In some embodiments, including that shown in FIG. 8A, the inflow edge of the atrial skirt portion 1210 may be positioned a spaced distance from the atrial tips of the atrial cells 1112. For example, in some embodiments that atrial end of the frame 1100 inflects towards the atrium (not shown in FIG. 8A), and thus outer sealing skirt 1200 may be terminated a spaced distance from the atrial end so that there is not a thrombogenic profile on the inflow end of the frame 1100. In other embodiments, the inflow edge of the atrial skirt portion 1210 may be positioned to align with or cover the atrial tips of the atrial cells 1112. It should be understood that the various tines 1126 preferably pierce through the sealing fabric 1200 so that the free ends thereof are available for frictional engagement with the native tissue upon implantation.

Still referring to FIG. 8A, the ventricular skirt portion 1220 may be more loosely connected to the ventricular portion 1120 of the frame 1100 than the atrial skirt portion 1210 is connected to the atrial portion 1110. For example, the outflow edge of the ventricular skirt portion 1220 may be relatively tightly coupled to the outflow end of the ventricular portion 1120 of the frame 1100, but the connection of the sealing skirt 1200 may be relatively loose between the central portion 1130 and the terminal end of the ventricular portion 1120 of the frame 1100. With this configuration, during ventricular systole (e.g., as the ventricle contracts, the prosthetic leaflets 1500 close, and the pressure in the ventricle is greater than the pressure in the atrium), the pressure differential causes the ventricular skirt portion 1220 to billow, inflate, or parachute open. As the ventricular skirt portion 1220 parachutes during ventricular systole, it may fill any gaps, crevices, or openings between the prosthetic heart valve 1010 and the native valve annulus that might otherwise result in blood leaking around the outside of the prosthetic heart valve 1010 back into the atrium (i.e., PV leak).

Referring briefly to FIG. 8D, the illustrated configuration of frame 1100 may provide a levering effect that may further assist with sealing against PV leak. For example, when the frame 1100 is in the expanded or deployed state shown in FIG. 8D, deformation of the ventricular portion 1120 may tend to lever the atrial portion 1110 toward the ventricular portion 1120. Thus, referring back to FIG. 8A, as the ventricular skirt portion 1220 inflates or parachutes during ventricular systole, which may cause the ventricular portion 1120 of the frame 1100 to slightly deform, the atrial portion 1110 of the frame 1100 may be lightly pulled downward against the atrial side of the native valve annulus. This “sandwiching” action may further seal against any PV leak, and may also mitigate potential embolization. For example, particularly in the low flow environment of the right heart, any gaps or spaces left between the prosthetic heart valve 1010 and the native anatomy may create a thrombus risk zone. The above-described levering or sandwiching effect may reduce or eliminate any such gaps or spaces, thus reducing the risk of thrombus formation. In one particular example, patients may have a pronounced septal bump, and some patients may have in particular a septal bump in the right ventricle that overhands the tricuspid valve annulus. This anatomy may be an exclusion criterion for a transcatheter prosthetic tricuspid valve replacement. However, the sandwiching or levering effect described above may allow for prosthetic heart valve 1010 to be implanted into patients who have relatively pronounced septal bumps.

Referring again to FIG. 8D, in the deployed or expanded condition of the frame 1100, the bottom struts of the enlarged transition cells 1117, to which the CAFs 1140 are connected, extend in the outflow direction substantially parallel to the central longitudinal axis of the prosthetic heart valve 1010. With this positioning, the CAFs 1140 may be positioned in alignment with, or nearly in alignment with, the smallest diameter portion of the frame 1100 at the central potion 1130. In other words, the CAFs 1140 of frame 1100 are effectively cantilevered. This cantilevering of the CAFs 1140, if no additional support is provided, may result in certain disadvantages. As explained above, the prosthetic leaflets 1500 are coupled to the CAFs 1140. As a result, during ventricular systole when the prosthetic leaflets 1500 are closed and pressure is applied in the ventricular-to-atrial direction, the CAFs 1140 and the struts of the enlarged transition cells 1117 to which the CAFs 1140 are attached may deflect radially inwardly toward each other. Although some amount of deflection may be desirable, the length of the CAFs 1140 (which may extend between about 20-30 mm from the central portion 1130) may be such that a risk of over-deflection may result. If the CAFs 1140 deflect too much during ventricular systole, the prosthetic leaflets 1500 may not coapt correctly, leading to inefficient valve functionality. Also, another disadvantage of large amounts of deflection of the CAFs 1140 is that the struts from which the CAFs 1140 extend may fatigue rapidly, possibly leading to failure of the frame 1100.

Other potential disadvantages may result if the CAFs 1140 of frame 1100 do not have additional support. For example, FIG. 9A illustrates prosthetic heart valve 1010 being deployed from a delivery device DD, with the ventricular portion 1120 and ventricular skirt portion 1220 having begun to self-expand. While the atrial portion 1110 remains within the delivery device DD, a lever type of effect may result in which the CAFs 1140 tend to splay radially outwardly as the prosthetic heart valve 1010 begins to deploy. If the CAFs 1140 are not separately supported, the CAFs 1140 may tend to splay to a position that is radially outward of the shape-set position. As a result of this splaying, the prosthetic leaflets 1500 may be pulled or stretched, which can be seen in the view of FIG. 9A. Even if this splaying occurs temporarily during delivery, the prosthetic leaflets 1500 (and/or the sutures connecting the prosthetic leaflets 1500 to the CAFs 1140) may be damaged, stressed, or otherwise weakened enough to cause a risk that the prosthetic leaflets 1500 may either not function correctly upon implantation, or even if the prosthetic leaflets 1500 function appropriately upon implantation, the longevity of the prosthetic leaflets 1500 may be reduced as a result of the stress during splaying of the CAFs 1140.

A third potential disadvantage may result if the CAFs 1140 of frame 1100 do not have additional support. FIG. 9B illustrates the ventricular side of prosthetic heart valve 1010, which lacks a CAF support member. In FIG. 9B, pressure is being manually applied to the ventricular portion 1120 in a way that generally mimics pressure that would result during ventricular systole (note that during actual ventricular systole, the prosthetic leaflets 1500 would close, which is not shown in FIG. 9B). Because the CAFs 1140 are connected to the ventricular portion 1120 of the frame 1100, deformation of the ventricular portion 1120 of the frame may result in deformation of the CAFs 1140, and particularly their positions relative to each other. As can be seen in FIG. 9B, as the ventricular portion 1120 of the frame 1100 is manually deformed the CAFs 1140 deform out of their generally circular or cylindrical alignment. This may be undesirable because as the CAFs 1140 deform away from their shape-set, generally circular or cylindrical alignment, the prosthetic leaflets 1500 become less likely to properly coapt with each other to form a seal.

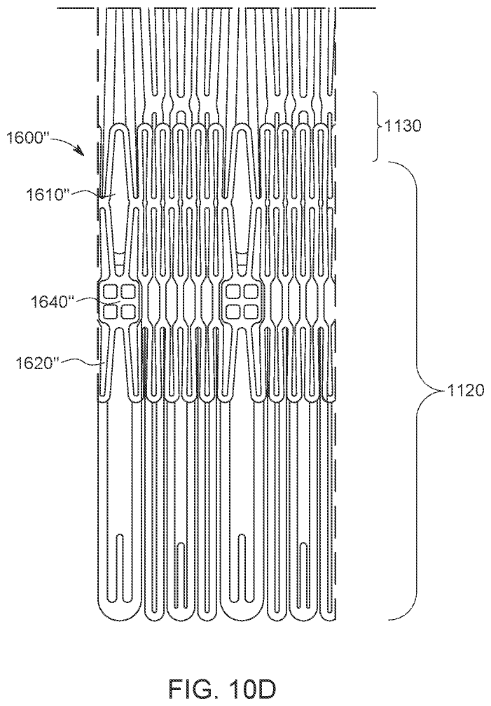

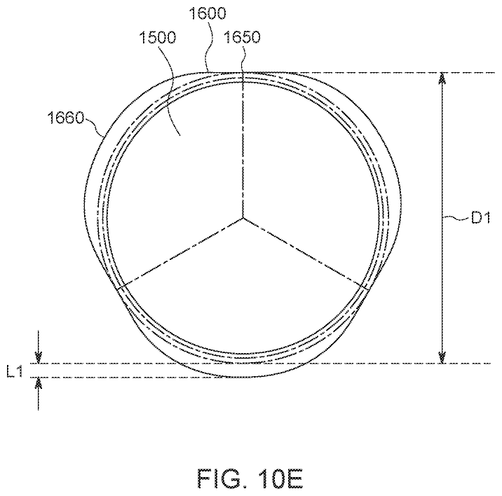

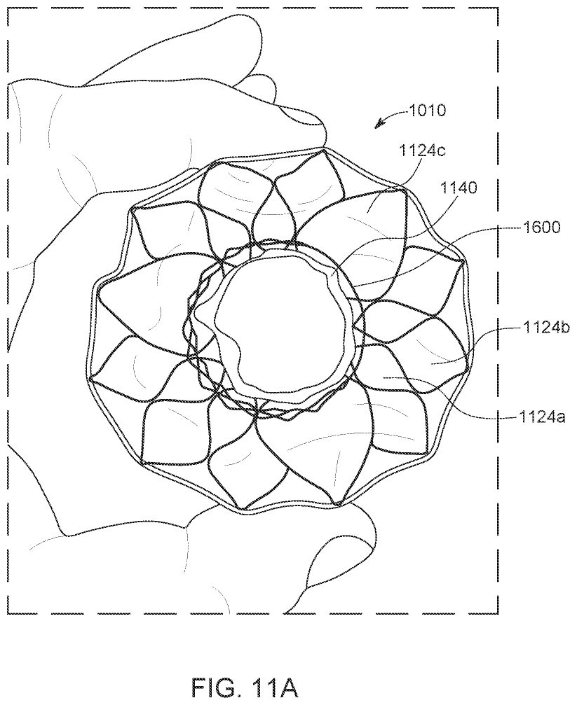

In order to address any one or more of the potential disadvantages of CAFs 1140 that exclude additional support members, a commissure support member 1600 (which may be referred to herein as a CAF support or simply a support member) may be provided. The CAF support 1600 is shown assembled to the frame 1100 in FIG. 8D. The CAF support 1600 may take various forms, but in some examples it may be an expandable and collapsible ring-shaped structure. FIG. 10A shows a cut pattern for one example of CAF support 1600. In the embodiment of FIG. 10A, the CAF support 1600 is formed of a shape-memory material, such as Nitinol, and may be laser-cut from a Nitinol tube using a pattern similar to that shown in FIG. 10A. The cut pattern shown in FIG. 10A is a single row of diamond-shaped cells 1610. In other words, after using the cut pattern of FIG. 10A on a tube of Nitinol, the resulting structure may be shape set (e.g., via heat treatment) so that, in the absence of applied forces, the CAF support 1600 forms a generally circular or cylindrical ring having a single row of diamond-shaped cells 1610. In the expanded or unbiased condition, the interior diameter of CAF support 1600 is about equal to the diameter of a circle that is aligned with the outer surfaces of the CAFs 1140 when the frame 1100 is in its expanded or unbiased condition.

The CAF support 1600 may be positioned on the exterior of the CAFs 1140 (and/or the cell struts from which the CAFs 1140 extend) and coupled to the frame 1100 via any suitable mechanism. For example, in some embodiments, the CAF support 1600 may be simply sutured to the CAFs 1140 and/or to the cell struts from which the CAFs 1140 extend. In other embodiments, either the CAF support 1600, the CAFs 1140 (or their associated struts), or both may include features to assist in the fixation. For example, referring back to FIG. 8C, one or both of the two struts that lead to the CAF 1140 may include one or more apertures 1142 that may be used to assist suturing the CAF support 1600 (e.g., at an intersection where two adjacent diamond-shaped cells 1610 meet) to the frame 1100. In the frames 1100 shown in FIGS. 8B-C, each strut leading to a CAF 1140 includes a single aperture at the same axial position. However, in the example shown in FIG. 8D, each strut leading to each CAF 1140 includes two apertures, and the apertures on one strut may or may not be axially aligned with the apertures on the other strut of the pair. It should be understood that the number, shape, and positioning of apertures 1142 may be other than that shown in the figures while still providing the desired functionality. And although suturing is described as one mechanism of fastening the CAF support 1600 to the frame 1100, it should be understood that other methods, such as adhesives, rivets (or other mechanical fasteners), etc. may be similarly suitable. Although CAF support 1600 is shown and described as being positioned on the exterior of the CAFs 1140, in some examples it may be positioned on the interior of the CAFs 1140.

FIG. 10B shows a cut pattern for a CAF support 1600′ that is slightly different from that of FIG. 10A. CAF support 1600′ may be similar or identical to CAF support 1600, with the exception that while CAF support 1600 includes a single row of diamond-shaped cells 1610, CAF support 1600′ includes two rows of diamond-shaped cells 1610′, 1620′. It should be understood that by varying parameters of the CAF supports 1600, 1600′ such as the size of the cells 1610, 1610′, 1620′, the wall thickness of CAF support, the width of the struts forming the cells, and/or the number of rows of cells in the CAF support, may all influence the performance of the CAF support. In other words, the size and collapsibility of the CAF support may be fine-tuned in part by varying these parameters, and the actual amount of support provided to the CAFs 1140 (which may determine the amount of splay-resistance, deformation, and/or deflection of the CAFs 1140) by the CAF support may be similarly fine-tuned in part by varying these parameters.

Although the CAF supports 1600, 1600′ are shown without any special features for assistance fixation of the CAF support to the frame 1100, it should be understood that such features may be provided on the CAF support 1600, 1600′. For example, apertures may be provided in the struts that form the CAF support 1600, 1600′ (e.g., at an intersection of two adjacent diamond-shaped cells) to facilitate fastening (e.g., suturing or riveting) the CAF support 1600, 1600′ to the frame 1100, whether or not the frame 1100 itself include similar (e.g., complementarily shaped, sized, or positioned) connection features.

In some embodiments, a CAF support may be configured to be directly coupled to the CAFs 1140, instead of or in addition to being coupled to cell struts that lead to the CAFs 1140. For example, FIG. 10C shows a cut pattern for a CAF support 1600″ that is slightly different from that of FIGS. 10A-B. CAF support 1600″ may be similar or identical to CAF support 1600′, with the main exception being that CAF support 1600″ includes an integrated connector 1640″ configured to facilitate direct coupling of the CAF support 1600″ to the CAFs 1140. For example, in the embodiment shown in FIG. 10C, the second row of cells 1620″ includes a connector 1640″ formed at the intersection of two adjacent diamond-shaped cells 1620″, with the connector 1640″ having a two-by-two configuration of apertures that has the same configuration as the two-by-two set of apertures in CAFs 1140. With this configuration, the CAF support 1600′ may be attached to the frame 1100 via an “overlay” technique, in which the connectors 1640″ are laid over the correspondingly shaped portions of CAFs 1140, and the four apertures are sewn (or otherwise coupled or mechanically fastened) directly over the corresponding portions of CAFs 1140. To help illustrate the complementary structure, FIG. 10D illustrates the cut pattern of the CAF support 1600″ overlaid on the cut pattern of the frame 1100. However, it should be understood that the CAF support 1600″ would generally be attached after the frame 1100 is expanded, and the CAF support 1600″ surrounds the CAFs 1140 (and struts attached to the CAFs 1140), but not the ventricular cells. It should be understood that, although one connector 1640″ is shown in FIG. 10C, a total of three connectors 1640″ would be provided to correspond to the three CAFs 1140 of frame 1100. If the frame 1100 included more or fewer than three CAFs 1140, a correspondingly similar number of connectors 1640″ may be provided on CAF support 1600″. Also, although a connector 1640″ is shown with a two-by-two configuration of apertures, other configurations may be provided. For example, if CAFs 1140 included a different configuration of apertures than shown in the figures, the connectors 1640″ may be provided with a correspondingly different configuration of apertures. And in some embodiments, the connectors 1640″ need not include the exact configuration of holes as the CAFs 1140, although such a one-to-one correspondence may simplify the coupling procedure. For example, even if CAFs 1140 include a two-by-two configuration of apertures (with or without the additional elongated eyelet), the connectors 1640″ could include a two-by-one or one-by-two set of apertures. One of the benefits of the configuration of support ring 1600″ is that it may simplify the process of coupling the support ring 1600″ to the frame 1100, and once coupled with the connectors 1640″ aligning in an overlay fashion with the CAFs 1140, the support ring 1600″ may be more likely to collapse and expand evenly and uniformly during loading into the delivery device and/or deployment from the delivery device.

The commissure support members shown and described above have been described as being shape-set or otherwise configured into a generally circular or cylindrical shape, which would generally match the shape of the perimeter of the CAFs 1140 when the prosthetic heart valve 1010 is expanded and/or deployed. However, in some cases, the prosthetic leaflets 1500 may open (e.g., during atrial systole) to an extent that would tend to extend radially outward of a circular perimeter formed along the CAFs 1140. In other words, if the commissure support 1600 (or 1600′ or 1600″) was formed as a circle and coupled to the outer surfaces of the CAFs 1140, the prosthetic leaflets 1500 might be at risk of contacting the inner surface of the commissure support when the prosthetic leaflets 1500 open. This type of contact would generally be undesirable. In order to mitigate this concern, any of the commissure supports described above, or any similarly suitable designs, may be shaped to provide clearance for the prosthetic leaflets 1500 when they open. For example, FIG. 10E shows a schematic of commissure support 1600 (although this may apply to the other commissure supports described herein) which, in its expanded condition, has a plurality of lobes. For example, in the given example, the prosthetic heart valve included three prosthetic leaflets 1500, and thus the commissure support 1600 is formed (e.g., via shape setting) to have three lobes. FIG. 10E shows that an outer diameter of the frame 1100 at the position of the outer surface of the CAFs 1140 of the frame 1100 has a value of D1. This outer diameter D1 at the CAFs may be, in one example, about 30 mm. Because the leaflets 1500 are coupled to the CAFs 1140, the leaflets 1500 at these locations do not change position (or at least not significantly) during opening and closing of the prosthetic leaflets 1500. Thus, at these locations the commissure support 1600 has a minimum extent 1650 that is in contact with the outer surface of the CAFs 1140. However, as the prosthetic leaflets 1500 open, the middle of the free edges is typically the portion that may have the greatest movement outwardly. In order to account for the possibly large movement, the commissure support 1600 increases in terms of radial extent from the longitudinal center of the prosthetic heart valve 1010 to a maximum extent 1660 that is positioned about midway along the perimeter of the commissure support 1600 between two adjacent minimum extents 1650. At this maximum extent 1660, the distance between the outer surface of the frame 1100 and the commissure support 1600 may have a length L1. Although the length L1 may be adjusted as desired, in one example the length L1 is about 1.5 mm. In other words, the middle of the free edges of the prosthetic leaflets 1500 may elongate up to about 1.5 mm beyond the diameter D1 without interference from the commissure support 1600. In a prosthetic heart valve 1010 with three leaflets 1500, the illustrated commissure support 1600 in FIG. 10E would have three minimum extents 1650 positioned about 120 degrees apart (aligned with the CAFs 1140), and three maximum extents 1660 positioned about 120 degrees apart (generally aligned with the middle of the free edges of the prosthetic leaflets 1500) with the maximum extents 1660 being offset by about 60 degrees from the minimum extents 1650. The particular shape of the transition between the minimum extents 1650 and maximum extents 1660 may vary, but preferably create a lobe shape similar to that shown in FIG. 10E to provide for a large amount of extra space for movement of the prosthetic leaflets 1500.

It should be understood that the extra clearance described above (e.g., the extra length L1) is not always necessary, and the particular design of the prosthetic leaflets 1500 may affect whether or not any interference might be expected with the use of a circular commissure support 1600. It should also be understood that the lobed design of commissure support 1600 may still provide some or all of the benefits described above in connection with a circular design of commissure support 1600, including mitigation of splaying, deformation, and/or over-deflection of the CAFs 1140.

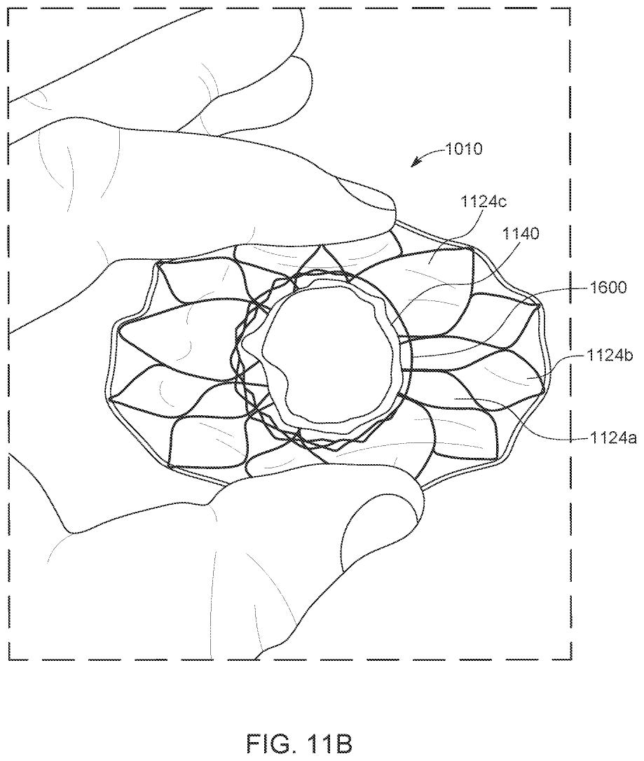

FIG. 11A shows prosthetic heart valve 1010, which includes commissure support 1600, prior to manual compression being applied to the ventricular portion 1120 of the frame 1100. In FIG. 11A, commissure support 1600 is shape-set to a circle, and not the lobed configuration of FIG. 10E. In the condition of FIG. 11A, both the commissure ring 1600 and ventricular portion 1120 maintain a highly circular profile. However, FIG. 11B illustrates the change after manual compression is applied to the ventricular portion 1120 of the frame 1100. As can be seen in FIG. 11B, the ventricular portion 1120 of the frame 1100 undergoes a significant amount of ovalization. In other words, the ventricular portion 1120 of the frame 1100 becomes oval-shaped in profile with a large discrepancy between the long and short axis of the oval profile after manual compression. Despite the ventricular portion 1120 of the frame 1100 becoming significantly ovalized, the commissure support 1600 (and thus the CAFs 1140) maintain an almost perfect circular profile. It should be understood that, although the commissure support 1600 in FIG. 11B may be slightly ovalized, the force being applied in FIG. 11B is larger than what would be expected when prosthetic heart valve 1010 is used as a prosthetic tricuspid valve, with the large amount of force being applied to illustrate the significant difference between the shapes of the ventricular portion 1120 of the frame 1100 and the commissure support 1600. When actually in use in a prosthetic heart valve (e.g., a prosthetic tricuspid valve), the commissure support 1600 may be expected to maintain a nearly perfect circular profile to maintain optimal coaptation of the prosthetic leaflets 1500.

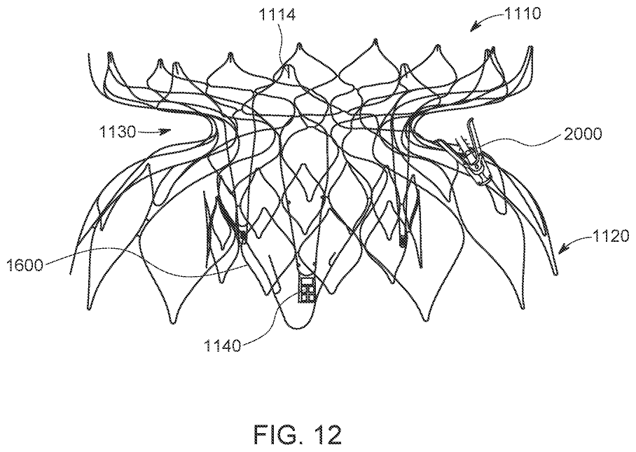

FIG. 12 shows the prosthetic heart valve 1010 (with the “soft” structure such as the prosthetic leaflets 1500 and sealing skirt 1200 removed for clarity) after having been implanted into a prosthetic heart valve that has already been treated previously with a leaflet repair clip 2000. It should be understood that the anatomy is also omitted from FIG. 12, showing only the relative position of the prosthetic heart valve 1010 and the already-existing repair clip 2000 to better show the relative positions of the devices. One advantage of the shape of frame 1100, including the shape of the central waist 1130 and/or the bell-shaped ventricular portion 1120, is that additional spaces may be left with respect to the native valve annulus to implant the prosthetic heart valve 1010 over a previously implanted leaflet repair clip 2000. The leaflet repair clip may be any device that clips two adjacent prosthetic leaflets 1500 together for and edge-to-edge type repair, such as the MitraClip™ device or TriClip™ valve repair devices offered by Abbott Laboratories to clip two mitral valve or tricuspid valve leaflets together, respectively. If the leaflet repair clip 2000 device was previously implanted an acceptable distance near the native leaflet commissure (e.g., similar to the relative position of the leaflet repair clip 2000 to the prosthetic heart valve 1010 shown in FIG. 12), the sealing skirt 1200 may be able to seal against PV leak, despite the obstruction caused by the leaflet repair clip 2000. And if the cells of the frame 1100 at the ventricular portion 1120 are large (e.g., cells 1124b, 1124c), the previously implanted leaflet repair clip 2000 may not exert any substantial pressure against the annulus as a result of the prosthetic heart valve 1010, since the leaflet repair clip 2000 may fall within the open area of one of these large cells.

As should be understood from the disclosure provided herein, in some embodiments, a prosthetic heart valve includes a single frame (e.g., a Nitinol frame) with a commissure support member to facilitate the prosthetic heart valve having a minimal profile with a wide treatment range of annular anatomy. The frame design and/or the commissure support member help to minimize the pressure of which the prosthetic heart valve exerts against the native valve annulus. The single layer frame helps to enable the prosthetic heart valve to be compressed inside of a catheter with a small diameter (e.g., <33 French or <30 French), with sealing achieved in part by a sealing fabric that spans the gap between the atrial and ventricular disks of the frame. The commissure support may help to ensure the long-term durability of the prosthetic leaflets and the ventricular portion of the frame. And while the disclosure provided herein may be applied to prosthetic heart valves for replacing mitral or tricuspid valves, these features may work particularly well with the tricuspid valve due to the lower ventricular pressures involved, which may reduce the need for a bulkier two-piece frame design. In addition, the native tricuspid valve does not have the more pronounced fibrous structure found in the native mitral valve. Thus, instead of utilizing the native structure surrounding the annulus (as is often done for a prosthetic mitral valve) that works well to handle the compression other prosthetic heart valves use, the prosthetic heart valve described herein may anchor within the tricuspid valve annulus via one or more of (i) light pressure or draping of the generally bell-shaped ventricular portion 1120 of the frame; (ii) ventricular tines 1126 providing frictional engagement with the native valve annulus; and/or (iii) parachuting of the sealing skirt 1200 assisting with fixation.

It should be understood that, although the prosthetic heart valve 1010 is described as including a frame 1100 and a separate commissure support 1600, the inclusion of the commissure support 1600 does not significantly increase the profile of the prosthetic heart valve 1010 when in the collapsed condition, compared to more traditional two-framed valves that may be used in mitral valve prostheses.

In an exemplary use of the prosthetic heart valves described herein, a prosthetic heart valve may begin in the expanded condition prior to implantation into a patient. As described above, the prosthetic heart valve may include a single monolithic or unitary stent with an outer fabric on the stent, with a commissure support member (either circular or lobed) circumscribing the commissure attachment features. The prosthetic heart valve may be drawn or otherwise forced into a delivery catheter, the prosthetic heart valve transitioning into the collapsed condition as it moves into the delivery catheter. Preferably, the outer diameter of the catheter of the delivery device has a size of 30 French (10 mm) or smaller, including 28 French (9.33 mm) or smaller or 24 French (8 mm) or smaller. With the prosthetic heart valve successfully collapsed in the small diameter delivery device catheter, the delivery device may be introduced into the patient, for example through the femoral vein, and navigated to the target site, for example the native tricuspid valve. Upon reaching the target site, the prosthetic heart valve may be deployed from the delivery device catheter, for example by retracting the delivery device catheter relative to the prosthetic heart valve. As the constraint on the prosthetic heart valve is removed, the prosthetic heart valve will naturally begin to expand as the stent tends to return to its preset shape. Preferably, the ventricular disk of the prosthetic heart valve is released first within the ventricle (e.g., the right ventricle). As the ventricular disk expands, the ventricular disk may begin to apply light pressure on the tissue, and the ventricular tines may frictionally engage (with or without piercing) the native tissue. If another device, such as tricuspid valve leaflet repair clip, has already been implanted previously, the prosthetic heart valve is preferably oriented so that some, most, or all of the leaflet repair clip is oriented in alignment with the open portion of a cell on the ventricular side of the frame. As deployment continues, the center portion of the stent of the prosthetic heart valve will generally align with the valve annulus. As the atrial side of the prosthetic heart valve deploys, the atrial disk of the stent will expand on the atrial side of the native valve. Thus, as described above, a small delivery device may be used, despite the requirement of coverage of a large native valve annulus area, and without losing any sealing capabilities despite using only a single stent with a small center portion housing the prosthetic leaflets.

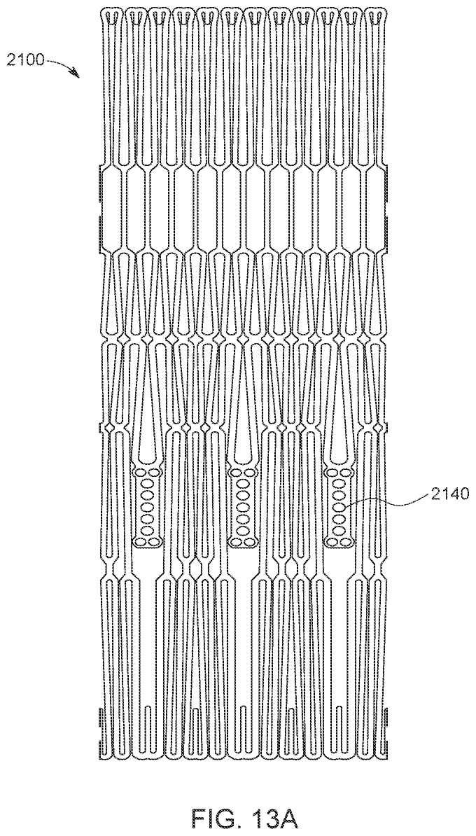

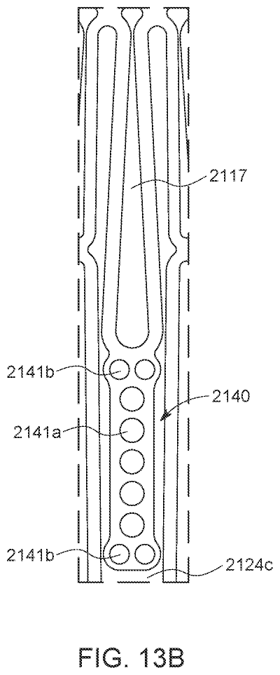

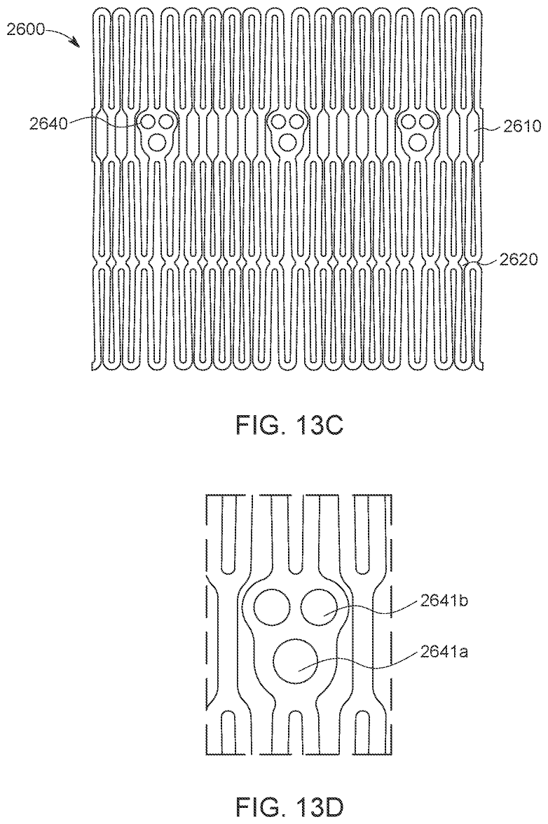

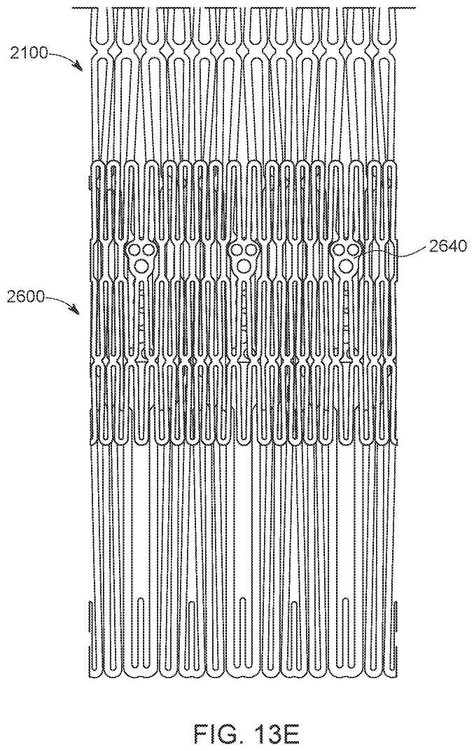

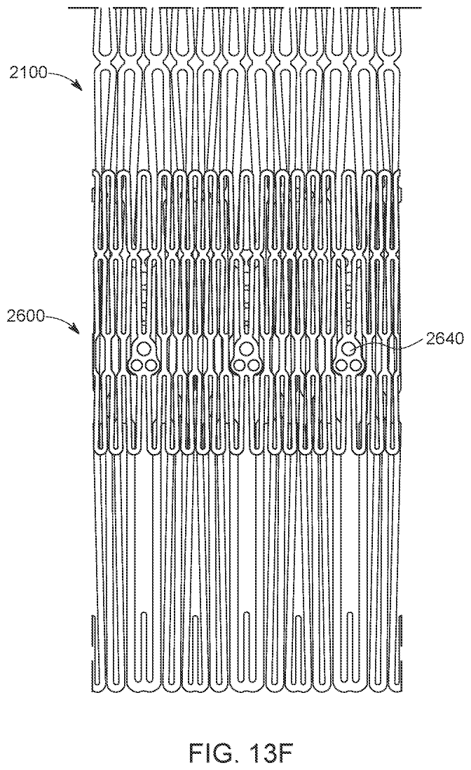

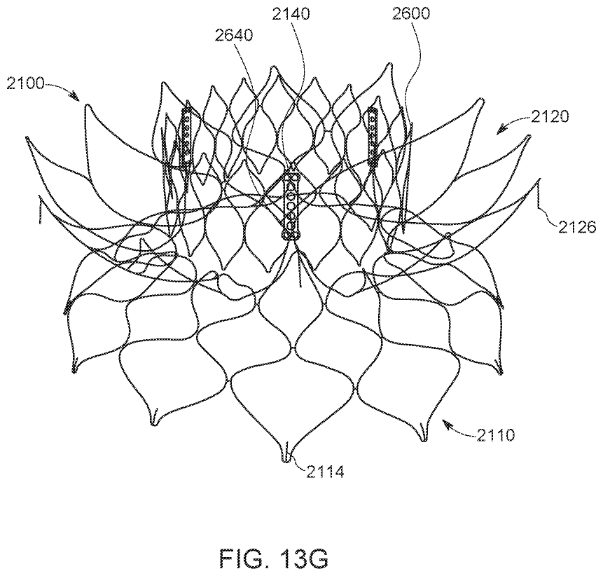

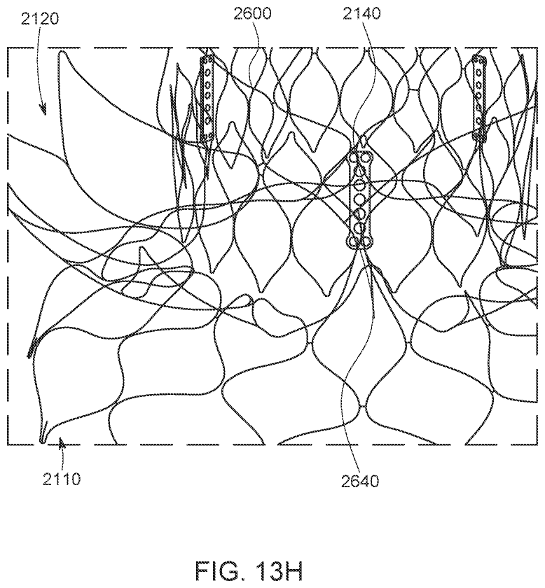

FIG. 13A shows a cut pattern of an alternate frame 2100 that is nearly identical to frame 1100 of FIG. 8C, with the main exception being the structure of CAFs 2140. Although frame 2100 may also include some other slight differences compared to the frame 1100 of FIG. 8C, such as the number and positioning of the ventricular tines, as well as the exact shapes of certain cells, only the CAFs 2140 of frame 2100 are described in greater detail below for purposes of brevity. Otherwise, the frame 2100 may be similar or identical to frame 1100, and the description of frame 1100 may otherwise apply to frame 2100.

FIG. 13B shows an enlarged view of one of the CAFs 1240. As with CAF 1140, CAF 1240 may be coupled to two struts at the outflow end of an enlarged transition cell 2117. Also, as with CAF 1140, CAF 1240 may either be thought of as forming a boundary of, or being nested within, ventricular cells 2124c. Each CAF 1240 may have a general beam-shape and may include a single column of eyelets 2141a, with the column of eyelets 2141a including a horizontal or circumferential row of two eyelets 2141b flanking the opposite ends of the column of eyelets 2141a. As a result, the CAF 2140 may have a general “dog-bone” shape. The column of eyelets 2141a may provide for multiple options for a stable attachment of the commissures of the prosthetic leaflets to the CAF 2140, and the general shape of the CAF 1240 does not foreshorten as the frame 2100 expands, as may occur with diamond-shaped features. The horizontal pairs of eyelets 2141b may help provide additional stability of the commissure tissue sutured through the eyelets 2141b in the axial or flow direction of the prosthetic heart valve.

FIG. 13C illustrates a cut pattern of an alternate version of a commissure support 2600. Commissure support 2600 may be generally similar to those described above, and the description of the other embodiments (including alternates described therewith) may generally apply to commissure support 2600. In the illustrated embodiment, commissure support 2600 includes a first row of cells 2610 and a second row of cells 2620, and integrated connectors 2640 provided on the commissure support 2600. The main difference between commissure support 1600″ and commissure support 2600 is the shape of the connectors 2640, which are shaped to be complementary to the shape of CAFs 2140. As shown in the enlarged view of FIG. 13D, the connector 2640 may have a partial “dog-bone” shape that includes a central eyelet 2641a and two eyelets 2641b arranged in a horizontal pair adjacent to the central eyelet 2641a. In this particular embodiment, the three eyelets of the connector 2640 may provide for a three-point connection to the corresponding CAF 2140. In particular, the two horizontally arranged eyelets 2641b may align with either pair of eyelets 2141b, with the central eyelet 2641a aligning with an eyelet 2141a in the column of eyelets of the CAF 2140 positioned adjacent to the relevant pair of horizontal eyelets 2141b.

The symmetry of the eyelets in the CAF 2140 may allow for the commissure support 2600 to be coupled to the frame 2100 in two different orientations. For example, FIG. 13E illustrates the commissure support 2600 overlaid on the frame 2100 with the connectors 2640 aligned with the inflow side of the CAFs 2140, whereas FIG. 13F illustrates the opposite orientation in which the connectors 2640 are aligned with the outflow side of the CAFs 2140. In either orientation, the commissure support 2600 may generally overlie the same portions of the frame 2100. In other words, either orientation of the commissure support 2600 may be used relative to the frame 2100 without any significant deviation in the resulting functionality, but it may be desirable to have different options for assembly. As explained in connection with FIG. 10D, the views of FIGS. 13E-F are only intended to illustrate how the commissure supports 2600 could overlay the CAFs 2140, and in the actual assembled and expanded condition, the commissure supports 2600 would only surround the CAFs 2140 and not the remaining ventricular cells of the frame.

The above point is illustrated in FIGS. 13G-H, which shows the frame 2100 and the commissure support 2600 in an assembled and expanded condition with other components of the prosthetic heart valve omitted from the views. In the view of FIG. 13G, the atrial (or inflow) portion 2110 (which may include pins 2114) is positioned toward the bottom of the view, and the ventricular (or outflow) portion 2120 (which may include tines 2126) is positioned toward the top of the view. The commissure ring 2600 is positioned radially inward of the ventricular portion 2120, other than the CAFs 2140 which are positioned within the commissure ring 2600. As can be seen in FIG. 13G, and particularly in the enlarged view of FIG. 13H, the connectors 2640 may be aligned with the CAFs 2140 at the inflow end of the CAFs 2140, which matches the first orientation shown in FIG. 13E. Although not shown in FIGS. 13G-H, a buffer material may be provided between the contact points of the commissure ring 2600 and the frame 2100, so that there is no or minimal direct metal-to-metal contact. Any buffer material may be suitable, including fabric materials or tissue materials, and similar buffer materials may be provided with other embodiments described herein to prevent or minimize metal-to-metal contact between a frame and a commissure support.

In some examples, a method of manufacturing a prosthetic heart valve for replacing a native atrioventricular valve includes forming a collapsible and expandable frame from a tube of shape-memory material. The method may include shape-setting the collapsible and expandable frame so that, in the absence of applied forces, the collapsible and expandable frame has an atrial disk, a ventricular disk, and a center portion extending between the atrial disk and the ventricular disk, the frame including a plurality of commissure attachment features that include struts that extend from the center portion of the frame. A plurality of prosthetic leaflets may be mounted to the plurality of commissure attachment features. A sealing fabric may be attached to an outer surface of the collapsible and expandable frame. After shape-setting the collapsible and expandable frame, a commissure support ring may be attached to the plurality of commissure attachment features to support the plurality of commissure features. The commissure support ring may be formed from a tube of shape-memory material. The commissure support ring may be shape-set to have a ring-shape or lobed-shape in the absence of applied forces. After shape-setting the collapsible and expandable frame, and after shape-setting the commissure support ring, in the absence of applied forces, the atrial disk and the ventricular disk may each flare outwardly from the center portion of the frame, the center portion of the frame may define a minimum diameter of the frame, and each of the plurality of commissure attachment features may be spaced from adjacent ones of the plurality of commissure attachment features so that gaps in the frame are present between adjacent ones of the plurality of commissure attachment features

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve for replacing a native atrioventricular valve, the prosthetic heart valve comprising:
a collapsible and expandable frame, the frame including an atrial disk, a ventricular disk, and a center portion extending between the atrial disk and the ventricular disk, the frame including a plurality of commissure attachment features that include struts that extend from the center portion of the frame;
a plurality of prosthetic leaflets mounted to the plurality of commissure attachment features;
a sealing fabric coupled to an outer surface of the frame; and
a commissure support ring coupled to and extending around the plurality of commissure attachment features,
wherein in an expanded condition of the prosthetic heart valve, the atrial disk and the ventricular disk each flare outwardly from the center portion of the frame, the center portion of the frame defining a minimum diameter of the frame, each of the plurality of commissure attachment features being spaced from adjacent ones of the plurality of commissure attachment features so that gaps in the frame are present between adjacent ones of the plurality of commissure attachment features.

2. The prosthetic heart valve of claim 1, wherein the commissure support ring is a collapsible and expandable structure that has a circular shape in an expanded condition of the commissure support ring.

3. The prosthetic heart valve of claim 1, wherein the commissure support ring is a collapsible and expandable structure that has a lobed shape in an expanded condition of the commissure support ring.

4. The prosthetic heart valve of claim 3, wherein in the expanded condition of the commissure support ring, first portions of the commissure support ring aligned with the plurality of commissure attachment features have a minimum diameter of the commissure support ring, and second portions of the commissure support ring aligned with middle portions of free edges of the plurality of prosthetic leaflets have a maximum diameter of the commissure support ring.

5. The prosthetic heart valve of claim 1, wherein the commissure support ring includes a first circumferential row of generally diamond-shaped cells.

6. The prosthetic heart valve of claim 5, wherein the commissure support ring includes a second circumferential row of generally diamond-shaped cells adjacent the first circumferential row.

7. The prosthetic heart valve of claim 6, wherein the commissure support ring includes a plurality of connectors integrally formed with the commissure support ring, the plurality of connectors each having a shape that is complementary to a shape of each of the plurality of commissure attachment features.

8. The prosthetic heart valve of claim 1, wherein the frame includes a plurality of tines on the ventricular disk, each of the plurality of tines extending to a free end pointing toward the atrial disk in a collapsed condition of the frame.

9. The prosthetic heart valve of claim 8, wherein in the expanded condition of the prosthetic heart valve, at least some of the plurality of tines extend at an acute angle relative to a central longitudinal axis of the prosthetic heart valve.

10. The prosthetic heart valve of claim 8, wherein in the expanded condition of the prosthetic heart valve, at least some of the plurality of tines extend at an obtuse angle relative to a central longitudinal axis of the prosthetic heart valve.

11. The prosthetic heart valve of claim 1, wherein the struts that extend from the center portion of the frame include at least one aperture.

12. The prosthetic heart valve of claim 11, wherein the commissure support ring is coupled to the plurality of commissure attachment features via mechanical fasteners extending through the at least one aperture.

13. The prosthetic heart valve of claim 1, wherein in the expanded condition of the prosthetic heart valve, the ventricular disk of the frame is bell-shaped.

14. The prosthetic heart valve of claim 13, wherein the sealing fabric extends over the ventricular disk and over the center portion of the frame, an inflow edge of the sealing fabric being positioned a spaced distance from a terminal end of the atrial disk.

15. The prosthetic heart valve of claim 13, wherein, in an implanted condition of the prosthetic heart valve, at least a portion of the sealing fabric is configured to parachute into contact with structure of the native atrioventricular valve during ventricular systole.

16. The prosthetic heart valve of claim 1, wherein the plurality of commissure attachment features extend from the center portion of the frame in an outflow direction, and the commissure support ring is positioned in the outflow direction relative to the center portion of the frame.

17. A method of implanting a prosthetic heart valve, the method comprising:
loading the prosthetic heart valve into a delivery device, the prosthetic heart valve including a collapsible and expandable frame having an atrial disk, a ventricular disk, a center portion extending between the atrial disk and the ventricular disk, a plurality of commissure attachment features that include struts that extend from the center portion of the frame, and a plurality of prosthetic leaflets mounted to the plurality of commissure attachment features;
advancing the delivery device to a native heart valve of a patient while the prosthetic heart valve is maintained in a collapsed condition by the delivery device;
while the delivery device is positioned in or adjacent to the native heart valve, starting to deploy the prosthetic heart valve in a ventricle of the patient so that the ventricular disk begins to expand and so that a sealing fabric coupled to an outer surface of the frame moves toward the native heart valve; and
continuing to deploy the prosthetic heart valve so that the center portion is positioned against an annulus of the native heart valve and the atrial disk expands within an atrium of the patient,
wherein while starting to deploy the prosthetic heart valve, a commissure support ring coupled to and extending around the plurality of commissure attachment features limits the distance which the plurality of commissure attachment feature can splay outwardly.

18. The method of claim 17, wherein while starting to deploy the prosthetic heart valve, at least one of a plurality of tines on the ventricular disk frictionally engage tissue of the native heart valve.

19. The method of claim 17, wherein the commissure support ring is a collapsible and expandable structure that has a circular shape after the prosthetic heart valve is fully deployed within the native heart valve.

20. The method of claim 17, wherein the commissure support ring is a collapsible and expandable structure that has a lobed shape after the prosthetic heart valve is fully deployed within the native heart valve.

21. The method of claim 20, wherein after the prosthetic heart valve is fully deployed within the native heart valve, first portions of the commissure support ring aligned with the plurality of commissure attachment features have a minimum diameter of the commissure support ring, and second portions of the commissure support ring aligned with middle portions of free edges of the plurality of prosthetic leaflets have a maximum diameter of the commissure support ring, such that as the plurality of prosthetic leaflets open during atrial systole, the middle portions of the free edges of the plurality of prosthetic leaflets reach a position radially outward of the first portions of the commissure support ring and radially inward of the second portions of the commissure support ring.
